(19) United States
(12) Patent Application Publication
(10) Pub. No.: US 2025/0369977 A1
(43) Pub. Date: Dec. 4, 2025

(54) Bio-Inspired Proline Sensors for Diagnosis and Surveillance of Stress in Living Systems

(71) Applicant: Northeastern University, Boston, MA (US)

(72) Inventors: Daniel J. Wilson, Medford, MA (US); Cassandra Martin, Wilmington, MA (US); Josephine R. Cicero, Burlington, MA (US); Audrey Moos, Boston, MA (US); Dorthea Geroulakos, Burlington, MA (US)

(21) Appl. No.: 19/228,723

(22) Filed: Jun. 4, 2025

Related U.S. Application Data
(60) Provisional application No. 63/655,991, filed on Jun. 4, 2024.

(51) Int. Cl. G01N 33/68; G01N 21/77; G01N 21/78; G01N 33/00

(52) U.S. Cl. CPC G01N 33/6812; G01N 21/78; G01N 33/0098; G01N 2021/7766

(57) ABSTRACT

Described herein, in some embodiments, are kits, devices and sensors comprising a conjugated aldehyde. In some embodiments, the sensors comprise an α,β-unsaturated aldehyde. Also described herein are methods of analyzing stress in a plant sample, the methods comprising: contacting the plant sample with a sensor or a device comprising the sensor; and comparing color intensity of the sensor or the device with a reference color intensity to determine stress level of the plant.

### RELATED APPLICATION(S)

This application claims the benefit of U.S. Provisional Application No. 63/655,991 filed on Jun. 4, 2024. The entire teachings of the above application(s) are incorporated herein by reference.

### GOVERNMENT SUPPORT

This invention was made with government support under Grant Number W911QY-19-9-0011 awarded by the US Army Combat Capabilities Development Command Soldier Center. The Government has certain rights in the invention.

### BACKGROUND

From decorative houseplants to the crops that feed the world, plants are subjected to a variety of environmental stresses over their lifetimes. Both local and global changes in climate, and factors like pollution and disease, can threaten the health status of plants, requiring time-sensitive interventions to prevent irreversible consequences including widespread crop losses.

### SUMMARY

To monitor plant health and maintain a sufficient supply of agricultural goods, existing tools for detecting environmental stresses to plants range from visual inspection to highly technical equipment. The most accessible strategies require advanced user training, while automated solutions requiring advanced technical infrastructure are typically limited to large-scale farming operations. In some embodiments, disclosed herein are a bio-inspired colorimetric sensing strategy and sensors for measuring proline, a ubiquitous biomarker of stress in plants. In some embodiments, signals generated by these sensors range from pale yellow, indicative of unreacted sinapaldehyde, to deep red, indicative of proline-dependent formation of a natural pigment called nesocodin. These devices may be used to differentiate between proline concentrations (e.g., concentrations ranging from about 0 mM to about 15 mM) in plant tissue. This approach highlights the opportunity to design field-deployable, user-friendly tools for agricultural monitoring, improved farming efficiency, and strengthened food security.

In one embodiment, disclosed herein is a sensor comprising a conjugated aldehyde.

In another embodiment, the sensor comprises an α,β-unsaturated aldehyde (e.g., sinapaldehyde).

In another embodiment, disclosed herein is a device comprising: a sensor of the present disclosure; and a porous wicking fabric.

In another embodiment, the device further comprises a substrate comprising cellulose, for example, wherein the substrate is coupled to the sensor and the fabric.

In another embodiment, disclosed herein is a kit, comprising: a sensor of the present disclosure or a device comprising the sensor; and an extraction solvent.

In yet another embodiment, disclosed herein is a method of determining proline concentration in a plant sample, the method comprising: contacting the plant sample with a sensor of the present disclosure or a device comprising the sensor; and determining the proline concentration based on color intensity of the sensor or the device.

In yet another embodiment, disclosed herein is a method of analyzing stress in a plant, the method comprising: contacting a sample of the plant with a sensor of the present disclosure or a device comprising the sensor; and comparing color intensity of the sensor or the device with a reference color intensity to determine stress level of the plant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A: An example of a three-sensor device before sample analysis. Along with a proline sensor (left), the other two sensors may serve as controls and may be utilized to determine if a device works correctly (middle) and if a sample is prepared correctly (right). For example, the pH sensor may be used to measure the pH of the sample and the hydration sensor may be used to check sample flow through the wick.

FIG. 1B: Example schematics of 1-, 2-, and 3-sensor devices. For example, the devices may be adjusted to hold a plurality of (e.g., three) paper-based sensors that may each be embedded with different materials. The devices may be assembled with similar alignment components or tools that are illustrated in FIG. 1C.

FIG. 1C: Example schematic of the layers of a one-sensor device. The paper-based sensors are prepared by aligning layers of polyethylene terephthalate (PET), fabric, and paper and holding them together with an adhesive on the top and bottom of the sensor. Layers 1, 4 and 7 are alignment tools used for the fabrication of the device but are not part of the final device.

FIG. 1D: Nesocodin synthesis with different bases. Absorbance spectra of nesocodin prepared in methanol from 300 to 700 nm where either the base or the concentration of the base was changed. The nesocodin prepared using tributylamine (sample in light grey) was synthesized according to the protocol by Roy et al.^{[56]} The molar ratio of sinapaldehyde to tributylamine was 2.6:1. Nesocodin was prepared with NaOH where the first sample (dark gray) was at a 2.6:1 molar ratio of sinapaldehyde to NaOH and the second sample (black) was at a 1:1 molar ratio of sinapaldehyde to NaOH.

FIG. 2: Nesocodin formation in methanol and water. Absorbance profiles from 300 to 700 nm of nesocodin synthesized in either methanol or water. Both solutions consisted of a 1:1:1 molar ratio of sinapaldehyde to proline to NaOH. The prominent peaks at 343 and 430 nm represent sinapaldehyde and deprotonated sinapaldehyde, respectively. In the spectrum where water is the solvent, the nesocodin peak is present at 505 nm, but in the spectrum where methanol is the solvent, the peak shifts to 520 nm.

FIGS. 3A-3F: Determination of nesocodin extinction coefficient. FIG. 3A: The plotted relationship between absorbance at 520 nm and nesocodin concentration when a first nesocodin solution was prepared in methanol. FIG. 3B: The plotted relationship between absorbance at 520 nm and nesocodin concentration when a second nesocodin solution was prepared in methanol. FIG. 3C: The plotted relationship between absorbance at 520 nm and nesocodin concentration when a third nesocodin solution was prepared in methanol. FIG. 3D: The plotted relationship between the absorbance at 505 nm and nesocodin concentration when a first nesocodin solution was prepared in water. FIG. 3E: The plotted relationship between the absorbance at 505 nm and nesocodin concentration when a second nesocodin solution was prepared in water. FIG. 3F: The plotted relationship between the absorbance at 505 nm and nesocodin concentration when a third nesocodin solution was prepared in water. For FIGS. 3A-3F, the solutions were prepared using a 1:1:1 molar ratio of sinapaldehyde to proline to NaOH. From the Beer-Lamber Law, the extinction coefficient of each solution is extrapolated from the slope of the best fit line (dotted line). The extinction coefficients are presented in Table 1.

FIGS. 4A-4D: Nesocodin formation in solution. FIG. 4A: Absorbance profiles of nesocodin from 300 to 600 nm formed in water with increasing molar ratios of sinapaldehyde to proline. The reaction is allowed to occur for 15 minutes before measuring the spectra. FIG. 4B: The peak intensities of the peaks at 343, 430, and 505 nm on each spectrum in FIG. 4A. FIG. 4C: Absorbance profiles from 300 to 600 nm of sinapaldehyde and another amino acid at a 1:25 molar ratio of sinapaldehyde to amino acid in water. FIG. 4D: Peak intensities at 343, 430, and 505 nm for the profiles in FIG. 4C. In all samples, NaOH is used as the base at a 1:1 molar ratio with sinapaldehyde.

FIGS. 5A-5B: Reaction between sinapaldehyde and amino acids in solutions. FIG. 5A: The absorbance profiles from 300 to 600 nm of the reaction between sinapaldehyde, an amino acid, and NaOH at a 1:1:1 molar ratio in water. The reaction was allowed to occur for 15 minutes before measuring the spectra. FIG. 5B: Peak intensities at 343, 430, and 505 nm for all the absorbance profiles in FIG. 5A.

FIGS. 6A-6D: Colorimetric responses of sinapaldehyde and isatin sensors based on amino acid identity and concentration. FIG. 6A: The colorimetric response of the sensors to six different amino acids at 0, 1, 10, 25, and 50 mM. Results are an average of 3 sensor replicates and error is reported as the standard deviation. FIG. 6B: Representative images of sensors with each amino acid at a low (1 mM), medium (10 mM), and high (50 mM) concentration. FIG. 6C: The colorimetric response of isatin-based sensors to six different amino acids at 0, 1, 10, and 50 mM. Results are an average of 3 sensor replicates and error is presented as the standard deviation. The legend for FIG. 6C is the same as the legend for FIG. 6A. FIG. 6D: Representative images of the isatin-based sensors with each amino acid at a low (1 mM), medium (10 mM), and high (50 mM) concentration.

FIG. 7: Color development in sensors over time. The effect of time on color development in the paper-based sensors at proline concentrations ranging from 0 to 50 mM, and samples are prepared in water. Results are an average of three sensor replicates and error is reported as the standard deviation.

FIG. 8: Color change of sensors in the RGB space. The effect of proline concentration on each channel in the RGB color space. ImageJ is used to isolate each channel and this information is utilized to identify the channel that was most sensitive to the sensors. Unlike in other figures, the y-axis is not normalized. Results are an average of three sensor replicates and error is the standard deviation.

FIG. 9: Colorimetric response of nesocodin sensors. The effect of proline concentration on the color of the nesocodin sensors at proline concentration ranging from 0 to 50 mM. The proline analyte samples are prepared in water and 50 mM of NaOH is added to make the solutions basic. Results are an average of three sensor replicates and error is reported as the standard deviation. The images are representative images of the sensors at 0, 1, 3, 5, 10, 20, 30, 40, and 50 mM proline.

FIG. 10: Calibration curve with isatin assay. A calibration curve where isatin was embedded into the WHATMAN® 5 chromatography paper in the same manner that the sinapaldehyde was in the paper-based sensors. Results are an average of three sensor replicates and error is reported as the standard deviation. The images above are representative images of the sensors at 0, 1, 3, 5, 10, 20, 30, 40, and 50 mM proline.

FIGS. 11A-F: Role of other amino acids in colorimetric signal development. FIG. 11A: The effect of alanine (Ala) on the colorimetric measurement of proline in the sensors. FIG. 11B: The effect of arginine (Arg) on the colorimetric measurement of proline in the sensors. FIG. 11C: The effect of aspartic acid (Asp) on the colorimetric measurement of proline in the sensors. FIG. 11D: The effect of glutamic acid (Glu) on the colorimetric measurement of proline in the sensors. FIG. 11E: The effect of leucine (Leu) on the colorimetric measurement of proline in the sensors. Samples that consisted of either a 1:3, 1:1, or 3:1 molar ratio of proline to the other amino acid are prepared. Each graph also includes the colorimetric response of either proline or the other amino acid at 0, 2.5, 5, 7.5, and 10 mM. The top images on each plot are of representative sensors used to measure a combination of proline and an additional amino acid at a 1:3, 1:1, and 3:1 molar ratio of proline to the other amino acid from left to right. The bottom images on each plot are representative images of the interfering amino acid alone at 2.5, 5, and 7.5 mM from left to right. FIG. 11F: A summary of the responses of all sample combinations and proline alone at 2.5, 5, and 7.5 mM. A two-tailed t-test with equal variance where p<0.05 was used to compare the proline control with each amino acid combination at each concentration. The asterisk (*) indicates that the signal from a sample containing a combination of proline and another amino acid significantly differs from the control with just proline. Results are an average of 3 sensor replicates and error is reported as the standard deviation.

FIGS. 12A-B: Effects of other amino acids on proline color response in sensors. FIG. 12A: Representative images of the paper-based sensors with different concentrations of the six different amino acids. FIG. 12B: Representative images of the paper-based sensors with combinations of proline and one of the other amino acids. Samples contained either 2.5 mM proline and 7.5 mM of another amino acid, 5 mM proline and 5 mM of another amino acid, or 7.5 mM proline and 2.5 mM of another amino acid.

FIG. 13: Effect of solvent on proline extraction. The extraction capabilities of two different solvents were tested: 3% (w/v) sulfosalicylic acid in water and 100% ethanol. Two leaves were removed from the same ornamental cabbage plant and an extraction protocol was performed with each solvent. The signal from the sample extracted with ethanol was approximately double that of the sample extracted with sulfosalicylic acid. There was also some color heterogeneity in the sulfosalicylic acid samples. Results are an average of three sensor replicates and error is reported as one standard deviation.

FIGS. 14A-14B: Effect of sucrose of nesocodin formation in sensors. FIG. 14A: The influence of increasing concentrations of sucrose on the nesocodin reaction and color formation in the sensors when samples were prepared in water. Proline concentration was kept at 10 mM and the NaOH concentration was kept at 50 mM. FIG. 14B: The effect of increasing concentration of sucrose on the color formation in the sensors when the proline samples were prepared in 3% (w/v) sulfosalicylic acid. Proline concentration was kept at 10 mM, but the NaOH concentration was increased to 0.25 M to ensure that the samples were basic. Results are the average of three sensor replicates and error is reported as the standard deviation.

FIGS. 15A-15C: Proline detection in ornamental cabbage plants. FIG. 15A: Representative image of the control and experimental ornamental cabbage plants before osmotic stress. FIG. 15B: Representative image of both cabbage plants after the experimental plant was watered with a salt solution. FIG. 15C: The colorimetric response of the sensors in response to plant stress. Samples were taken from both plants before and after generating osmotic stress in the experimental plant. Collected samples after the stress event were divided—half were frozen and the other half were dried. The proline was later extracted with ethanol and measured with the sensors. Results are an average of three sensor replicates and error is the standard deviation. The images are representative images of the sensors with each sample condition. For statistical analysis, a two-tailed t-test was performed with equal variance with a p-value of <0.05. Significance of the sensor results for the control and experimental plant for each condition (e.g. before stress, frozen, and dried) was analyzed individually and the asterisk (*) indicates that the results are significantly different. Although the colorimetric signals of the control and experimental plants were significantly different from each other, both results indicated that there was an undetectable level of proline in the sample. Since biological samples are present, it is unsurprising that the two cabbage plants have slight differences in their proline content, and the difference does not affect the conclusions.

FIGS. 16A-16C: Detecting proline levels in kale plants. FIG. 16A: Representative images of control and stressed kale plants, depicting the plants before the experimental plant was subjected to thermal stress (top), after the first 24-hour heat treatment (middle), and after the second 24-hour heat treatment (bottom). Scale bars are approximately 50 mm. FIG. 16B: Calibration curve of green channel pixel intensity as a function of analyte concentration for sensors treated with proline solutions prepared in ethanol. The trendline was created using a four-parameter fitting function resulting in an R² value of 0.995. Results are an average of 3 sensor replicates and error is the standard deviation. FIG. 16C: Proline concentrations after each stress event for both plants. The proline concentration of each sample was calculated using the calibration curve in FIG. 16B. Results are an average of 3 sensor replicates and error is the standard deviation. A two-tailed t-test was performed with equal variance and a p-value <0.05 for statistical analysis comparing the control and experimental plant after each stress event. Statistically different results are denoted with an asterisk (*). Images are representative images of the sensors for each condition.

FIGS. 17A-17C: Top view of ornamental kale plants. FIG. 17A: The top view of the ornamental kale plants before the stress events. FIG. 17B: The top view of the ornamental kale plants after the first stress event. FIG. 17C: The top view of the ornamental kale plants after the second stress event. The control plant (left) was kept under a grow light at room temperature and the experimental plant (right) was exposed to 35° C., 40% humidity conditions for 24-hour periods. Outer leaves from both plants were removed after the first stress event (after FIG. 17B was taken but before FIG. 17C to measure proline levels. The scale bars are 2 inches.

FIGS. 18A-18B: Colorimetric response in sensors as a product of analyte solvent. FIG. 18A: Representative images of sensors where the proline analyte was prepared in either water or ethanol. FIG. 18B: The calibration curves for water (from FIG. 9) and ethanol (from FIG. 16B) are overlaid on each other to compare the effect of the solvent on the color intensity. For both calibration curves, the results are the average of three sensor replicates and error is one standard deviation.

FIG. 19: Expanded sensor calibration curve. The calibration curve for the sinapaldehyde sensors where the proline analyte was prepared in ethanol. The graph is an expansion of the calibration curve in FIG. 16B, where 60, 70, 80, and 90 mM proline data points were included. Results are an average of three sensor replicates and error is the standard deviation. The images above are representative images of the sensors at the noted proline concentrations.

FIG. 20: Ninhydrin calibration curve. A calibration curve for detecting proline with the acid ninhydrin assay. Proline standards were prepared in 3% (w/v) sulfosalicylic acid and the path length was 0.5 cm. The equation for the best fit line is y=2.7623x+0.0052 and the R² value is 0.9975. The results are an average of three prepared standards at each concentration and error is reported as standard deviation.

FIG. 21: Plant stress analysis with the ninhydrin assay. The acid ninhydrin assay was used in parallel with the sensors to support the results of the sensors in the proof-of-concept experiment with the ornamental kale plants. The control kale plant was kept at room temperature and the experimental kale plant was exposed to 35° C., 40% humidity for 24 hours for each stress event. Proline was extracted from the plants with 3% (w/v) sulfosalicylic acid and the concentrations were concentrated from the calibration curve in FIG. 20. Leaves were sampled from both plants after each event and one solution of extract was prepared. Three acid ninhydrin assays were performed from this one stock and the results were averaged. Error is reported as the standard deviation of the concentrations.

FIGS. 22A-22C: Confirming sensor specificity using biological samples. FIG. 22A: Representative images of control and experimental green cabbage plants before the stress event. FIG. 22B: Representative images of the control and experimental green cabbage plants after the experimental plant was heated to 35° C. for 24 hours to initiate a stress response. Scale bars are approximately 13 mm. FIG. 22C: Measured proline concentrations of the control and experimental plants after the stress event, as well as measured concentrations of the experimental extract with the addition of 2, 6, and 10 mM exogenous proline. Results are an average of 3 sensor replicates and error is the standard deviation. Statistical analysis was performed using a one-way ANOVA test where the p-value was 0.05, followed by a Tukey test to determine which conditions were significantly different (indicated by the *).

FIGS. 23A-23D: Example self-contained microfluidic device design. FIG. 23A: The fully assembled device features a high-flow wicking material that delivers sample fluid to the sensor. Wicking fabric is exposed at the end of the device so that the device can be activated by dipping into a liquid sample matrix. After activation, the device rapidly fills with fluid, hydrating the sensor. FIG. 23B: Exploded schematic of additively assembled device layers. FIG. 23C: Representative images of a dry-stored device and activated devices measuring increasing concentrations of proline. FIG. 23D: Representative images of sample extracted from green cabbage plants using ethanol in a 5 mL centrifuge tube and devices used to measure stress responses of green cabbage plants. Scale bars are 9 mm.

FIGS. 24A-24C: Effect of different example device features on sensor color uniformity. FIG. 24A: A device with WHATMAN® 5 filter paper as the wick. FIG. 24B: A device without the WHATMAN® 3 filter layer between the absorbent fabric wick and the sensor. FIG. 24C: A device with both the fabric wick and the filter layer. Each device was tested with a 3 mM proline solution in ethanol. Scale bar is 9 mm.

### DETAILED DESCRIPTION

A description of example embodiments follows.

To accommodate expansion of the world's population, estimates project that global food production will need to increase by up to 62% in the coming decades.[1] However, changes in average regional temperatures[2] and water availability[2b, 3] have had detrimental effects on agricultural production as a result of climate change.[4] Additionally, global elevation of atmospheric carbon dioxide, and subsequently atmospheric temperature,[5] have been demonstrated to compromise the quality of grain crops. [6] Rather than cultivate new farmland to combat crop losses attributed to environmental factors, biomass balance models have indicated that improved farming efficiency on existing farmland could sufficiently increase agricultural yields. [7] Importantly, this approach does not require deforestation, preserving trees to combat climate change and protect biodiversity.[8] Beyond climatic variation, other challenges including pests[9] and disease[10] can cause significant crop losses that make it difficult for farmers to maintain or furthermore increase food production.[11] These factors will continue to threaten global food security in the coming decades.

New technologies for monitoring the health status of crops present an opportunity to detect threats to agricultural yields and make corrective interventions before sustaining crop losses. Advancements in “smart farming” have enabled data-driven decisions in crop monitoring and maintenance, [12] with hyperspectral, [13] multispectral, [14] and thermal[15] imaging techniques offering measurements of changes in plant health status before crops present visually obvious symptoms of damage or injury.[16] These sensors have been miniaturized and incorporated into handheld devices to allow for ease-of-use and portability for small-scale crop analysis,[16-17] and coupled to unmanned aerial systems to capture data-dense images for large-scale crop analysis.[18] While quantitative tools based on imaging or in vivo electrical measurements[19] offer farmers the ability to frequently obtain large quantities of data describing the health of their crops and land, reduce their workload, and make informed decisions to mitigate crop loss,[12, 18d, 20] they may not match the scale, operational capabilities, or financial constraints of smaller farms,[16, 21] including family farmers in industrialized countries or farmers in the developing world. The tradeoffs connecting cost, analytical performance, and practical implementation of tools designed to improve farming efficiency have limited their ultimate utility for a large population of the world's farmers.

Plants have multiple physiological markers for indicating environmental stress including changes in chlorophyll concentration,[22] reactive oxygen species,[23] total free amino acids,[24] and proline. Proline is a biomarkers used to monitor plant health, accumulating in plant tissue in response to stresses such as drought,[24b, 25] the presence of excessive salts[26] or heavy metals,[27] temperature extremes,[24a, 28] UV radiation,[29] xenobiotics,[30] and pathogens. [31] When a plant is under duress, proline is reported to perform several critical functions to counter these stressful stimuli to mitigate potential damage.[32] For example, proline is classified as an osmolyte,[33] meaning that it can aid in maintaining cell volume and turgor,[34] stabilize proteins,[35] support ion homeostasis,[26b] and act as a cryoprotectant.[36] Proline is also noted to chelate metals,[27b] neutralize reactive oxygen species,[37] maintain NADP+/NADPH levels,[38] and support cellular signaling.[32a] Further, plants provided with exogenous proline have demonstrated improved tolerance to various stresses.[39] As a result, monitoring proline concentrations in plant tissue is a practice for diagnosing plant stress, but standard measurement protocols have required spectrophotometers[40] or other specialized equipment in centralized laboratories.[41]

There are two common colorimetric assays for quantifying proline that have been used in not only the diagnosis of plant stress,[40a, 42] but also in the analysis of beverages,[43] protein,[44] and blood.[45] The ninhydrin assay is perhaps the most universal technique for measuring proline levels in plants. In this assay, ninhydrin and an amino acid react to form a Schiff base, and then the product undergoes a decarboxylation and condensation reaction to form the visible chromophore known as Ruehmann's purple.[46] The primary drawback of this mechanism is that color formation is not specific to proline, though acidic reaction conditions have been demonstrated to prevent the interaction between ninhydrin and amino acids with primary amine functional groups, resulting in improved selectivity.[47] Many iterations of this assay were developed over several decades, each highlighting how previous protocols were susceptible to the presence of interfering amino acids (e.g. glutamine,[48] lysine,[47b] and glycine[49]).[50] Protocols that employ acidified ninhydrin to detect proline from plant samples, used from 1973 to today, [26a, 40a] are limited by nonspecific interactions with interfering amino acids[40a] and inhibition by sugars in the sample matrix[51] to qualitative comparisons of stressed and unstressed plants. These protocols require organic solvents[52] like toluene and high temperatures (100-150° C.)[40a, 53] to drive chromophore formation. Protocols based on the colorimetric reaction between proline and isatin, another popular strategy for measuring proline, resulting in the formation of pyrrole blue, follow a similar experimental design but are cited as less susceptible to nonspecific interactions with other amino acids or hydroxyproline.[40b, 54] However, isatin assays also require high temperatures to drive color formation,[40b, 42, 44] are impacted by the presence of sugars,[43] and produce a light-sensitive product that can be degraded in as little as one hour.[44]

In efforts to translate these laboratory-based assays to rapid diagnostic tools that can be used on-location, both colorimetric chemistries have been incorporated into paper-based microfluidic device formats.[42, 53, 55] While this approach enables interpretation of colorimetric signals by visual inspection to provide semi-quantitative results, standard colorimetric strategies for detection of proline still rely on temperatures exceeding 100° C. to drive signal development, requiring the use of a portable heating apparatus[42, 53, 55] or restricting analysis to a laboratory setting.

Disclosed herein, in some embodiments, is a paper-based sensor that performs a colorimetric measurement of proline concentration based on the synthesis of a natural, plant-based pigment called nesocodin at room temperature. Nesocodin is a dark red pigment that was recently discovered as the primary colorant in the nectar of the Nesocodon mauritianus flower, created by formation of an imine bond between proline and sinapaldehyde to initiate pollination by other species via visual signaling. [56] The effects of different variables on nesocodin synthesis (e.g., alkalizing agents, solvents, amino acid reactants) were investigated herein in order to design a sensing scheme compatible with both aqueous and organic proline extraction matrices. The example sensors disclosed herein were prepared by embedding sinapaldehyde the sensing agent in the device—in paper-based substrates and their responses to proline over a biologically-relevant concentration range were quantified. While these sensors do not provide exclusive molecular specificity to proline, they exhibited differentiated color formation in response to proline over other amino acids present in plant sample matrices. As disclosed herein, these example sensors were packaged into simple microfluidic devices that autonomously delivered plant tissue extract to the sensors, enabling rapid, on-site detection of stress in real plant samples. Using a bio-inspired design strategy based on the pollination mechanism of flowers, these sensors eliminate requirements for equipment, laboratory infrastructure, and user training, enabling in-field plant stress diagnosis to improve farming efficiency, track crop health as a function of environmental variation, or investigate intentional efforts to damage agricultural goods (e.g., agricultural terrorism).

### User Experience & Outcomes:

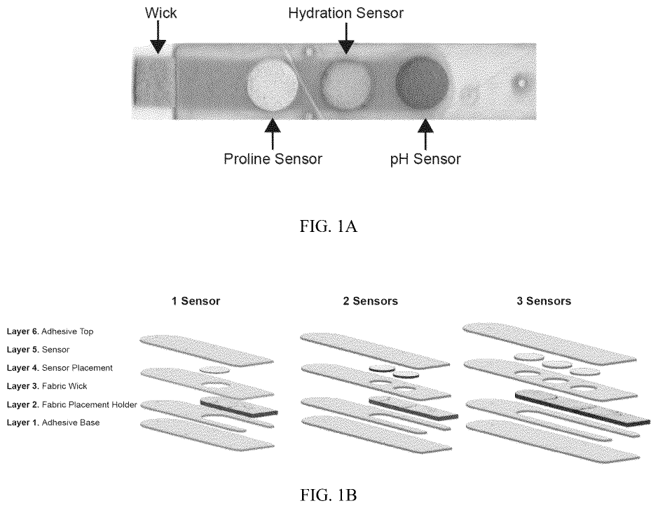

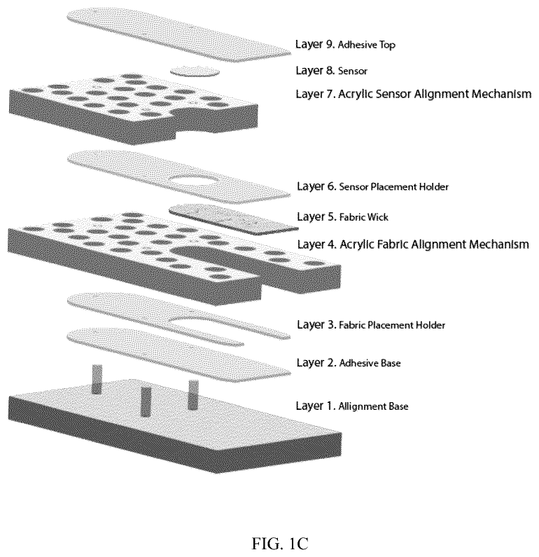

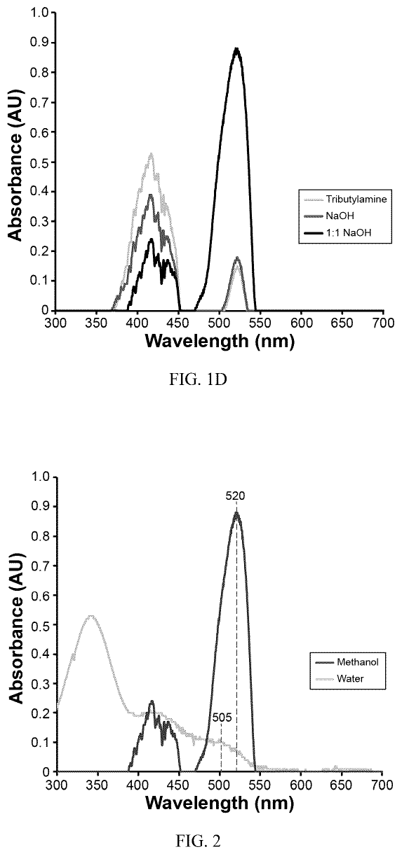

One advantage to the example device design provided herein is that the device can be customized to hold complementary paper-based sensors in order to provide the user with additional information. Along with the sensor embedded with sinapaldehyde to detect proline, the design can be customized to hold up to two additional sensors that can also be prepared on WHATMAN® 5 paper (FIGS. 1A and 1B). One sensor is a water detector sensor which is composed of cobalt chloride that has been embedded in the paper. This sensor serves as a quality control check that ensures the user that the device is working properly because it is positioned after the proline sensor. The pH sensor contains a universal pH indicator that is orange in the device before sample application. Nesocodin synthesis can only occur in a basic environment, so it is critical to validate that the sample is at the correct pH.

The present disclosure provides, in some embodiments, sensors comprising a conjugated aldehyde. In some embodiments, the conjugated aldehyde is an α,β-unsaturated aldehyde. Such aldehydes include, for example, cinnamaldehyde, and sinapaldehyde, coniferaldehyde.

In some embodiments, the aldehyde is

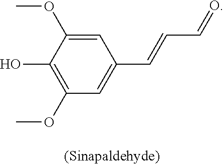

In some embodiments, the sensor is a paper-based sensor (e.g., WHATMAN® 5 paper-based sensor). The sensor may be based on one or more types of paper including but not limited to WHATMAN® filter paper, nitrocellulose paper, chromatography paper, and glass fiber paper. In some embodiments, the sensor further comprises cellulose. In some embodiments, the sensor comprises paper, e.g., a paper disc.

In some embodiments, the sensor has a pore size of less than about 10 μm (e.g., less than about 9 μm, less than about 8 μm, less than about 7 μm, less than about 6 μm, less than about 5 μm, less than about 4 μm, less than about 3 μm, less than about 2 μm, etc.). In some embodiments, the sensor has a pore size of less than about 6 μm. In some embodiments, the sensor has a pore size of less than about 2.5 μm. In some embodiments, the sensor has a pore size of about 2.5 μm.

In some embodiments, the sensor has a pore size of from about 0.1 μm to about 10 μm (e.g., about 0.1 μm to about 10 μm, about 0.1 μm to about 10 μm, about 0.1 μm to about 9 μm, about 0.1 μm to about 8 μm, about 0.1 μm to about 7 μm, about 0.1 μm to about 6 μm, about 0.1 μm to about 5 μm, about 0.1 μm to about 4 μm, about 0.1 μm to about 3 μm, about 0.1 μm to about 2.5 μm, etc.). In some embodiments, the sensor has a pore size of from about 0.1 μm to about 3 μm. In some embodiments, the sensor has a pore size of from about 0.5 μm to about 3 μm. In some embodiments, the sensor has a pore size of from about 0.7 μm to about 3 μm. In some embodiments, the sensor has a pore size of about 2.5 μm.

In some embodiments, the sensor has a diameter or a lateral dimension of less than about 100 mm (e.g., less than about 100 mm, less than about 90 mm, less than about 80 mm, less than about 70 mm, less than about 60 mm, less than about 50 mm, less than about 40 mm, less than about 30 mm, less than about 20 mm, less than about 10 mm, less than about 1 mm, etc.). In some embodiments, the sensor has a diameter or a lateral dimension of less than about 10 mm. In some embodiments, the sensor has a diameter of less than about 10 mm.

In some embodiments, the sensor has a diameter or a lateral dimension of from about 0 mm to about 100 mm (e.g., about 0 mm to about 90 mm, about 0 mm to about 80 mm, about 0 mm to about 70 mm, about 0 mm to about 60 mm, about 0 mm to about 50 mm, about 0 mm to about 40 mm, about 0 mm to about 30 mm, about 0 mm to about 20 mm, about 0 mm to about 10 mm, etc.). In some embodiments, the sensor has a diameter or a lateral dimension of from about 0 mm to about 10 mm. In some embodiments, the sensor has a diameter or a lateral dimension of from about 1 mm to about 100 mm (e.g., about 1 mm to about 90 mm, about 1 mm to about 80 mm, about 1 mm to about 70 mm, about 1 mm to about 60 mm, about 1 mm to about 50 mm, about 1 mm to about 40 mm, about 1 mm to about 30 mm, about 1 mm to about 20 mm, about 1 mm to about 10 mm, etc.). In some embodiments, the sensor has a diameter or a lateral dimension of from about 1 mm to about 10 mm. In some embodiments, the sensor has a diameter of from about 1 mm to about 10 mm. In some embodiments, the sensor has a diameter of about 9 mm.

In some embodiments, the sensor has a thickness of from about 0.1 mm to about 10 mm (e.g., about 0.1 mm to about 9 mm, about 0.1 mm to about 8 mm, about 0.1 mm to about 7 mm, about 0.1 mm to about 6 mm, about 0.1 mm to about 5 mm, about 0.1 mm to about 4 mm, about 0.1 mm to about 3 mm, about 0.1 mm to about 2 mm, about 0.1 mm to about 1 mm, etc.). In some embodiments, the sensor has a thickness of from about 0.1 mm to about 1 mm.

The present disclosure also provides, in some embodiments, devices comprising a sensor of the present disclosure. In some embodiments, a device comprises a sensor of the present disclosure and a porous wicking fabric. In some embodiments, a device comprises: a sensor of the present disclosure; and a hydration sensor, a pH sensor, or a combination thereof. In some embodiments, a device comprises a sensor of the present disclosure, a hydration sensor, and a pH sensor. In some embodiments, a device comprises a sensor of the present disclosure, a hydration sensor, a pH sensor; and a porous wicking fabric. In some embodiments, the porous wicking fabric comprises a base (e.g., sodium hydroxide).

Examples of a device of the present disclosure are illustrated in FIGS. 1A-C, 23B, and 24C.

In some embodiments, the porous wicking fabric comprises chamois, rayon, cotton, linen, polyester, silk, velvet, or a combination thereof. In some embodiments, the porous wicking fabric comprises rayon. In some embodiments, the wicking fabric is a high flow material with an open pore structure. In some embodiments, the wicking fabric exhibits a porosity consistent with, i.e., comparable to, a chamois cloth, e.g., a chamois-like commercial absorbent cloth, such as SHAMWOW® cloth.

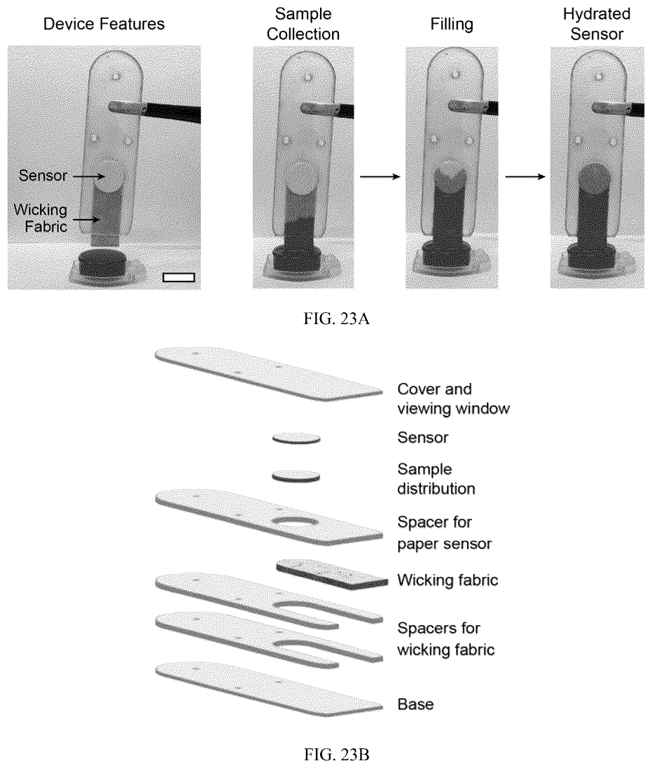

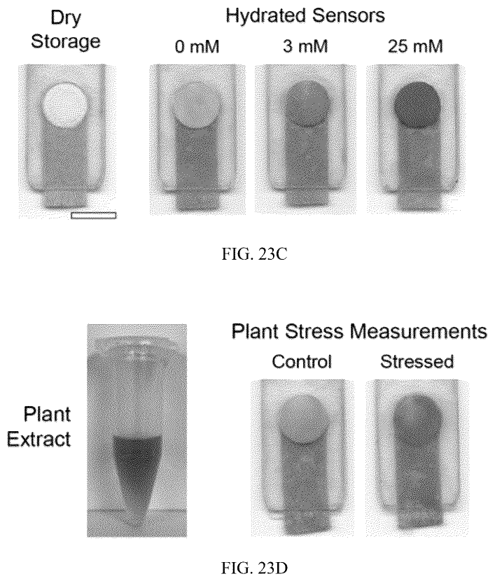

In some embodiments, a device of the present disclosure further comprises a substrate (e.g., a substrate for sample distribution) comprising cellulose, wherein the substrate is coupled to the sensor and the fabric. An example of such a device is illustrated in FIG. 23B. In some embodiments, the substrate (e.g., WHATMAN® 3 paper) has a larger pore size than the sensor. In some embodiments, the substrate has a pore size of larger than about 1 μm (e.g., larger than about 2 μm, larger than about 3 μm, larger than about 4 μm, etc.). In some embodiments, the substrate has a pore size of larger than about 3 μm. In some embodiments, the substrate has a pore size of about 6 μm.

In some embodiments, the substrate has a pore size of from about 2 μm to about 10 μm (e.g., about 3 μm to about 10 μm, about 4 μm to about 10 μm, about 4 μm to about 9 μm, about 4 μm to about 8 μm, etc.). In some embodiments, the substrate has a pore size of about 6 μm.

In some embodiments, a device of the present disclosure further comprises a cover. An example of a device comprising a cover is shown in FIG. 23B.

In one embodiment, a device of the present disclosure is a component in a kit, e.g., a test kit for detecting proline on-site. For example, a user may source the plant sample, perform the extraction with liquid components, and add the sample to the device, which may then be analyzed by, e.g., eye or camera, e.g., a smartphone camera. To reduce the burden of some of these steps for the user, in some embodiments, steps may be built into the device itself and it may be made more automated. In some embodiments, a porous wicking fabric comprises a base (e.g., sodium hydroxide). Incorporating a base into the wicking fabric may help initiate the nesocodin reaction and improve user convenience.

In some embodiments disclosed herein are kits, comprising a sensor of the present disclosure or a device of the present disclosure; and an extraction solvent.

Various extraction solvents are contemplated herein, including solvents comprising sulfosalicylic acid (e.g., 3% (w/v) sulfosalicylic acid in water), ethanol (e.g., 100% ethanol), trichloroacetic acid, perchloric acid, phosphate buffer, acetic acid, methanol, formic acid, and dilute hydrochloric acid.

In some embodiments, the extraction solvent comprises ethanol or sulfosalicylic acid. In some embodiments, the extraction solvent comprises ethanol. In some embodiments, the extraction solvent comprises sulfosalicylic acid. In some embodiments, the extraction solvent comprises water.

In some embodiments, the kit further comprises a solution comprising sodium hydroxide. In some embodiments, the sodium hydroxide has a concentration of from about 1 mM to about 500 mM (e.g., about 10 mM to about 500 mM, about 10 mM to about 400 mM, about 10 mM to about 300 mM, about 10 mM to about 200 mM, about 10 mM to about 100 mM, etc.). In some embodiments, the sodium hydroxide has a concentration of about 250 mM. In some embodiments, the sodium hydroxide has a concentration of about 50 mM NaOH.

In some embodiments, a kit or device of the present disclosure further comprises one or more of: a cutting tool (e.g., scissors), a grinding tool (e.g., grinder, such as, or similar to, a spice grinder), and/or a reference tool (e.g., a color chart, calibration curve information, etc.). An example of a reference tool is a color comparison chart, which allows a user to visually compare the color of a sensor (such as a test strip) or a device with predefined color standards. By comparing the color of the sensor or the device to the chart, for example, a user may determine the proline concentration or plant stress level.

### Methods

The present disclosure provides, in some embodiments, methods of determining an analyte concentration in a plant sample, the methods comprising: contacting the plant sample with a sensor of the present disclosure or a device comprising the sensor; and determining the analyte concentration based on color intensity (e.g., normalized color intensity) of the sensor or the device. In some embodiments, the analyte comprises an amine (e.g., primary amine, secondary amine, tertiary amine). In some embodiments, the analyte is an amino acid (e.g., proline). In some embodiments, the color intensity of the sensor is the red channel intensity (e.g., normalized red channel intensity). In some embodiments, the color intensity of the sensor is the blue channel intensity (e.g., normalized blue channel intensity). In some embodiments, the color intensity of the sensor is the green channel intensity (e.g., normalized green channel intensity).

In some embodiments, disclosed herein are methods of determining proline concentration in a plant sample, the methods comprising: contacting the plant sample with a sensor of the present disclosure or a device comprising the sensor (e.g., a device of the present disclosure); and determining the proline concentration based on color intensity of the sensor or the device.

As used herein, “plant sample” and “a sample of a plant” refer to plant-derived material in solution (e.g., plant material dissolved or suspended in a solvent such as ethanol) or plant-derived material not in solution (e.g., in dried form). The plant-derived material may include, but is not limited to, plant-derived molecules such as amino acids and sugars, plant cells, plant tissue, a leaf, part of a leaf, part of a stem, a root segment, a flower, a seed, a fruit, or any other plant tissue, whether fresh, dried, ground, or otherwise processed, and whether collected from cultivated or wild sources. For example, a plant sample may comprise of grounded (e.g., manually grounded) plant leaves and an extraction solvent (e.g., 100% ethanol, 3% (w/v) sulfosalicylic acid).

In some embodiments, a plant sample comprises plant material at a concentration of from about 0.01 g/mL to about 5.0 g/mL (e.g., about 0.01 g/mL to about 4.0 g/mL, about 0.01 g/mL to about 3.0 g/mL, about 0.01 g/mL to about 2.0 g/mL, about 0.01 g/mL to about 1.0 g/mL, about 0.1 g/mL to about 1.0 g/mL, etc.). In some embodiments, a plant sample comprises plant material at a concentration of about 0.5 g/mL.

In some embodiments, a plant sample is prepared by suspending plant material in a solvent (e.g., an extraction solvent) to form a solution, mixing the solution, and separating the plant material from the solution. In some embodiments, a base is added to the solution to adjust the pH of the solution. In some embodiments, sodium hydroxide (e.g., 50 mM NaOH) is added to the solution to adjust the pH of the solution. Various techniques for mixing a solution are contemplated herein, including vortexing, stirring, shaking, sonication, agitation, blending, and gentle inversion. Plant material may be separated from the solution by pushing the plant material to the sides of a container, pipetting the solution into a centrifuge tube, and using centrifugation to separate sedimented plant material from the solution. In some embodiments, the plant sample comprises an extraction solvent. In some embodiments, the plant sample further comprises sodium hydroxide.

In some embodiments, the plant sample has a pH of from about 7.1 to about 7.5 (e.g., about 7.1 to about 7.3). In some embodiments, the plant sample has a pH of about 7.2.

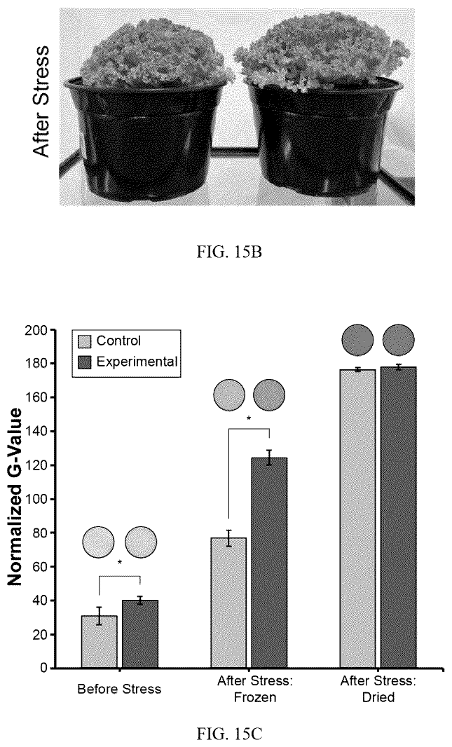

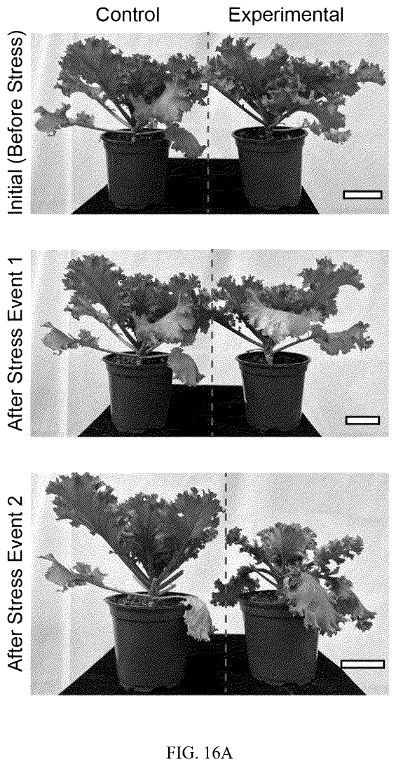

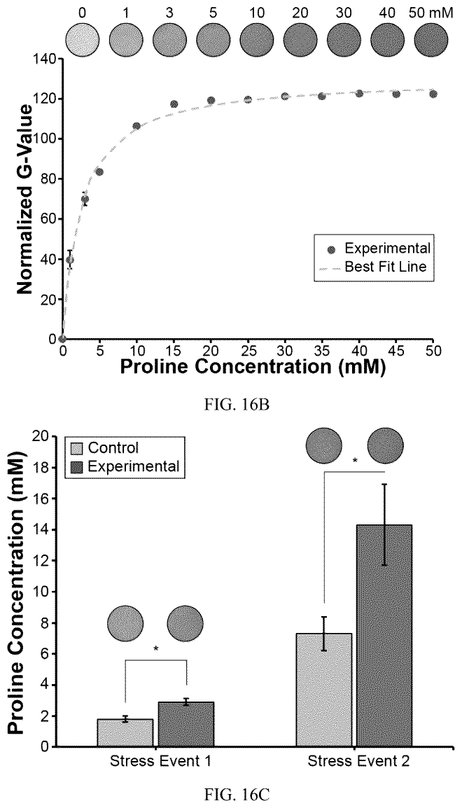

In some embodiments, determining the proline concentration based on color intensity of a sensor or a device of the present disclosure comprises comparing the color intensity of the sensor or the device with a reference tool (e.g., a calibration curve, a color comparison chart, etc.). In some embodiments, the reference tool is a calibration curve (e.g., a calibration curve obtained using known concentrations of proline and its pixel intensity). For example, the calibration curve may be prepared by measuring the green channel (G-value) pixel intensity from images of the sensor or device, e.g., based on the intensities of nesocodin formed at known concentrations of proline. Images of the sensor or device may be captured by a digital camera, smartphone, scanner (e.g., photo scanner), microscope, or other imaging equipment. The pixel intensity may be normalized by subtracting the color intensity of each sample from the color intensity (e.g., average color intensity) of the control. Based on a calibration curve (such as a logistic best-fit curve), the concentration of proline may be determined based on the color intensity of the sensor or the device. An example calibration curve is shown in FIG. 16B.

The present disclosure also provides, in some embodiments, methods of analyzing stress (e.g., thermal stress, osmotic stress) in a plant, the method comprising: contacting a sample of the plant with a sensor of the present disclosure or a device comprising the sensor (e.g., a device of the present disclosure); and comparing color intensity of the sensor or the device with a reference color intensity to determine stress level of the plant.

In some embodiments, the reference color intensity is obtained from a control (e.g., plant without stress). In some embodiments, the color intensity is green channel (G-value) pixel intensity of the sensor or the device. In some embodiments, comparing color intensity of the sensor or the device with a reference color intensity comprises performing a statistical analysis to determine the statistical significance of the difference in the color intensity of the sensor or the device and the reference color intensity. For example, if the difference in the color intensity of a test sample (e.g., measured in replicates) and the reference (e.g., a control or another test sample) is statistically significant, then the test sample indicates the plant is under stress (e.g., with respect to a control) or under greater stress (e.g., with respect to another test sample).

In some embodiments, statistical analysis includes one or more of t-test, ANOVA (Analysis of Variance), or non-parametric test. For example, if the p-value obtained from statistical analysis is below a predetermined threshold (e.g., 0.05), the difference is considered statistically significant and indicates the plant is under stress.

In some embodiments, stress is biotic stress (e.g., an antigen). In some embodiments, stress is abiotic stress (e.g., salt). Stress can, in some examples, be any of the stresses disclosed herein. In some embodiments, the stress is the result of tampering (e.g., poison).

In some embodiments, after a stress is determined and/or measured, measures are taken to intervene. For example, if the stress is caused by an antigen, affected plants may be killed to prevent stress in other plants. In another non-limiting example, if the stress is caused by high salt, additional watering of the plants may occur. Therefore, in some embodiments, the methods further comprise steps to reduce, ameliorate or remove the stress, or damage from the stress, or to prevent further stress or stress-medicated, for example, in the tested plants or other plants. Therefore, in some embodiments, disclosed herein are methods of reducing stress damage in plants comprising analyzing the stress using methods, samples and devices disclosed herein and then taking measures to alleviate or remove the stress or stress damage.

### Example Features and Advantages of Example Embodiments

Example sensors disclosed herein rely on a natural red pigment that has no known environmental hazards. The reaction between sinapaldehyde and proline leads to the formation of nesocodin, which is a natural colorant found in Nesocodon nectar and not harmful to the environment. Additionally, in the methods disclosed herein, there is minimal use of organic solvents compared to gold-standard colorimetric assays for proline determination.

In addition, in some embodiments, the devices disclosed herein are accessible for anyone to use in any location (e.g., in the field). They can be designed to be easily used by people with little to no formal scientific training, providing actionable information within minutes. An example device is easy to handle and interpret, requires no external equipment, and provides results in under 15 minutes.

The reaction in example sensors disclosed herein between sinapaldehyde and proline occurs quickly at room temperature, and, therefore, requires no external energy source to generate the results. This differs from other paper-based proline sensors that require high temperatures to initiate the chemical reaction. Further, since example sensors disclosed herein require no external energy/power source to initiate the chemical reaction, they can be used to measure the health status of plants without transporting samples back to a laboratory. Therefore, example embodiments do not require the use of expensive equipment or external power sources or power supply, nor do they require laboratory infrastructure or sample transportation.

Additionally, in some embodiments herein, the materials and reagents needed to construct example sensors and devices are inexpensive. In some embodiments, the sensors disclosed herein are prepared from inexpensive materials (e.g., paper, plastic, fabric, and/or double-sided tape), meaning that they are inexpensive to fabricate.

Additionally, methods of detection using the sensors do not require sample purification or analyte extraction with hazardous chemicals (e.g., toluene). The example sensors and devices are low hazard, low cost, and accessible for any person to use and interpret.

### Example Uses of Example Embodiments

The sensors and devices disclosed herein can be applied for use as a plant stress indicator. Researchers have shown that both biotic and abiotic stresses trigger proline accumulation in plants in order to alleviate or protect against the stress. The sensors and devices disclosed herein can be utilized as an on-site diagnostic tool for measuring stress in plants, including crops, and providing rapid feedback to the user.

The sensors and devices disclosed herein can be applied for use as a food quality control tool, for example, for quality control tests to assess the quality of food such as processed foods. In some embodiments, the foods are, for example, wine and honey. Winemakers use proline levels in their product to assess nitrogen content, wine type, and overall quality of their product. Additionally, the International Honey Commission requires a specific quantity of proline in honey; proline concentrations out of this range are considered to be either low quality or adulterated. In some embodiments, the methods, sensors and devices are used to test for tampering.

The sensors and devices disclosed herein can be applied to human and animal health and clinical diagnostics. For example, psychological stress in farm and agricultural animals can be tested. Correlation of proline concentrations to health status is not limited to plant biology. The concentrations of proline and related metabolites in human blood serum have been correlated to the presence of esophageal and other cancers.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.” The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element or step not specified in the claim; and (iii) “consisting essentially of” limits the scope of a claim to the specified materials or steps “and those that do not materially affect the basic and novel characteristic(s)” of the claimed disclosure and disclosure. Embodiments described in terms of the phrase “comprising” (or its equivalents) also provide as embodiments those independently described in terms of “consisting of” and “consisting essentially of”

“About” means within an acceptable error range for the particular value as determined by one of ordinary skill in the art, which will depend in part on how the value is measured or determined, i.e., the limitations of the measurement system. Unless explicitly stated otherwise within the disclosure, claims, result or embodiment, “about” means within one standard deviation per the practice in the art, or can mean a range of ±20%, ±10%, ±5%, ±4, ±3, ±2 or +1% of a given value. It is to be understood that the term “about” can precede any particular value specified herein, except for particular values used in the Examples.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLES

### Example 1

### Nesocodin Experiments in Solution

To establish design criteria for colorimetric sensors based on formation of nesocodin, we first explored several factors that impact synthesis of this natural colorant in solution. We began with a strategy for synthetic nesocodin preparation established during the discovery of the mechanism of color formation in Nesocodon mauritianus (Scheme 1). In this work, the authors formed a stable imine bond between sinapaldehyde and proline in the presence of a base catalyst, tributylamine, to create nesocodin.[56] Targeting reaction conditions and reagents compatible with devices and protocols that could be used on-location for interrogation of plant health, we first explored whether tributylamine, a hazardous and volatile material, could be replaced with sodium hydroxide (NaOH) to allow nesocodin to form in methanol. We demonstrated that different basic reagents produced solutions with similar absorbance profiles, including absorbance peaks at 430 nm and 520 nm (FIG. 1D). Previous results identifying a peak at 430 nm as deprotonated sinapaldehyde, activated to react with proline, and a peak around 500 nm corresponding to nesocodin aligned well with our preliminary results.[56] From these experiments, we concluded that tributylamine is not required for nesocodin formation, and the reaction can be supported by general basic conditions. Next, we increased the amount of NaOH in the reaction so there was a 1:1:1 molar ratio of sinapaldehyde to proline to NaOH, compared to a 2.6:2.6:1 ratio in the previous experiment. We determined that the additional NaOH further promoted nesocodin formation, which we attribute to more quantitative deprotonation of sinapaldehyde, creating more opportunities for imine bond formation between sinapaldehyde and proline.

Scheme 1. Nesocodin synthesis. The synthesis of the red pigment nesocodin occurs between sinapaldehyde and proline in a basic environment. In this reaction, the alcohol group on sinapaldehyde is first deprotonated by the base. This allows the aldehyde group of sinapaldehyde to react with the secondary amine of proline via a condensation reaction, forming a stable imine bond between the two reactants.^{[56]}

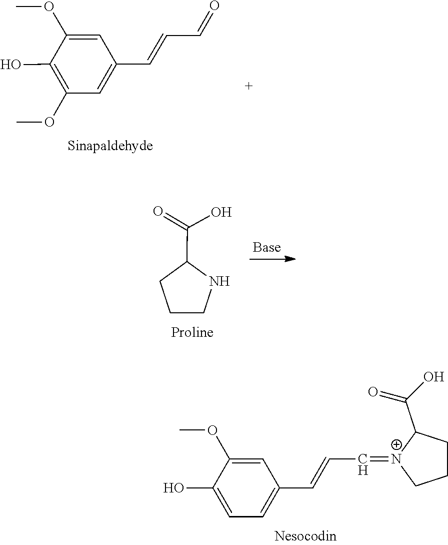 [SMILES: COc1cc(/C=C/C=O)cc(OC)c1O ; O=C(O)C1CCCN1 ; COc1cc(/C=C/C=[N+]2CCCC2C(=O)O)ccc1O]

Interestingly, the suspected nesocodin peak in these samples appeared at 520 nm, whereas in prior investigations of synthetic nesocodin, the product absorbed strongly at 505 nm.[56] We hypothesized that this difference may be due to differences in the absorptive properties of nesocodin prepared in the aqueous solutions (e.g., HEPES, Tricene, or MES buffers)[56] used in the previous study, and the methanol used in our analysis. To explore the dependence of the absorptive properties of the chromophore on the synthesis environment, we prepared stock solutions of sinapaldehyde and proline in water and mixed them in a 1:1:1 molar ratio with NaOH. In the resulting absorbance spectrum, we clearly observed that the nesocodin peak shifted to 505 nm, which further supports our hypothesis (FIG. 2). Additionally, we measured the molar extinction coefficients of nesocodin in methanol (at 520 nm) and water (at 505 nm) (Equation 1).

$$A = ε lc . \qquad Equation 1$$

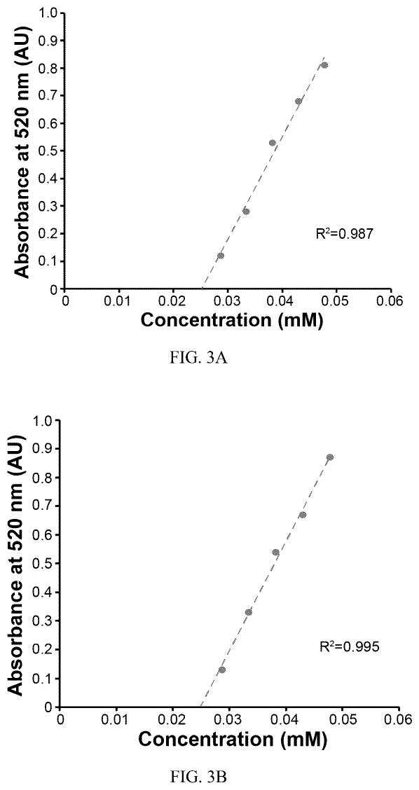

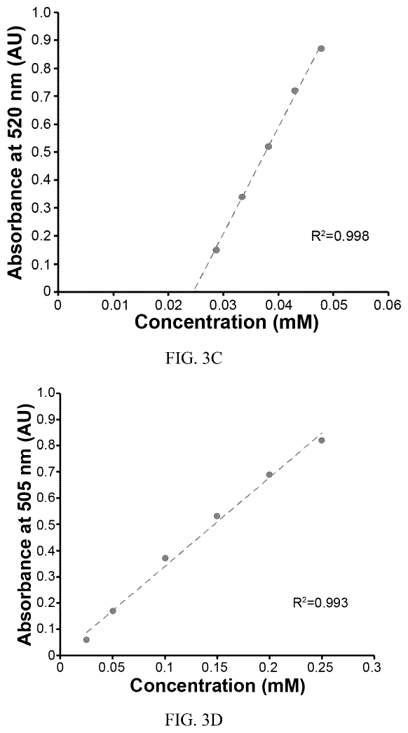

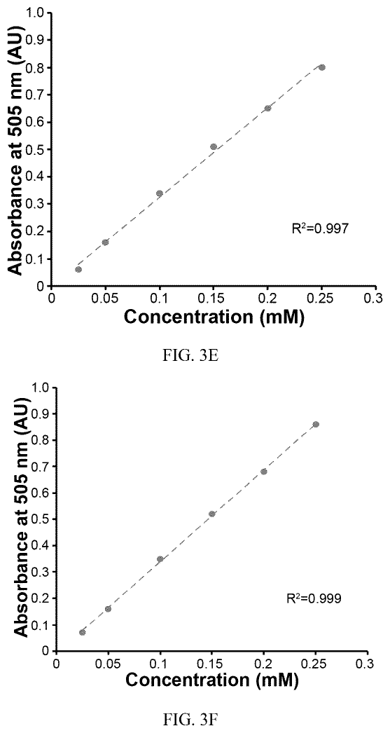

The extinction coefficient of nesocodin in methanol is 37824.7±483.8 M⁻¹ cm⁻¹ and 3381.9±108.5 M⁻¹ cm⁻¹ in water (FIGS. 3A-F and Table 1), indicating that nesocodin absorbs light approximately ten times as strongly in methanol as it does in water.

TABLE 1
Extinction coefficients of nesocodin
prepared under varying conditions.

| | | | Extinction | Standard |
|---|---|---|---|---|
| | | Main Peak | Coefficient | Deviation |
| Solvent | Base | (nm) | (M⁻¹ cm⁻¹) | (M⁻¹ cm⁻¹) |
| Methanol | NaOH | 520 | 37824 | ±483.7 |
| Water | NaOH | 505 | 3381 | ±108.5 |

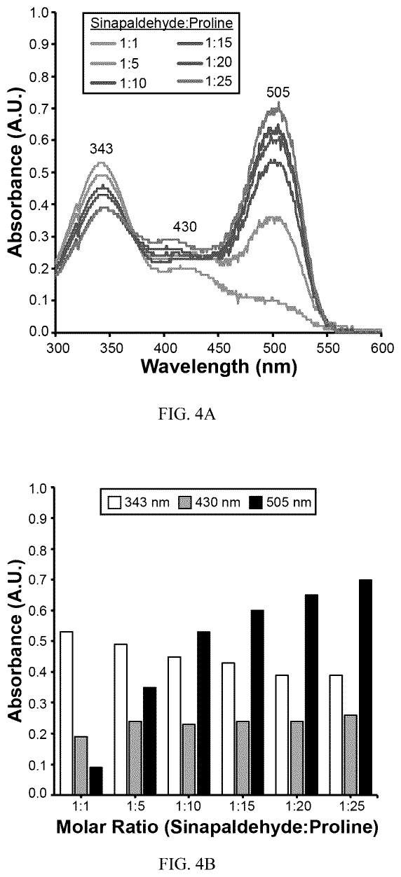

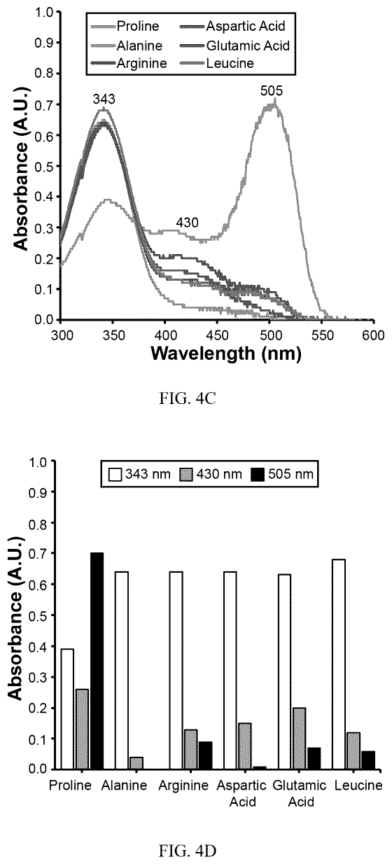

We next chose to further explore the nesocodin synthesis reaction in water. Apart from differences in the primary wavelength of the nesocodin peak, one prominent discrepancy between the absorbance profiles of nesocodin prepared in methanol versus water was the presence of a peak at 343 nm (FIG. 2), which has previously indicated the presence of protonated sinapaldehyde.[56] In our evaluation of these solvent conditions, we allowed both reactions to occur for the same amount of time. The differences between these spectra suggested limited transformation of sinapaldehyde in aqueous environments, indicating that the reaction did not proceed as efficiently in water as in methanol. We attempted to promote more nesocodin formation by increasing the molar ratio of proline to sinapaldehyde in the reaction mixture. We observed an increase in the absorbance of the nesocodin peak at 505 nm and a decrease in the sinapaldehyde peak at 343 nm, indicating that a molar excess of proline enhances nesocodin formation in a defined reaction period. It is interesting to note that apart from the 1:1 condition, the peak intensity at 430 nm remained approximately the same as the concentration of proline increased (FIGS. 4A and 4B).

Sinapaldehyde Reaction with Other Amino Acids

A major challenge with existing assays for proline detection is balancing the complexity and requirements of the test protocol with specificity for the target analyte. One drawback of the ninhydrin assay—the most common technique for quantifying proline—is that it is not specific to proline, meaning that interfering species are typically removed via chromatography before analysis.[40a, 48, 50] Our next step was to investigate the specificity of sinapaldehyde for detecting proline by determining if other amino acids initiate chromophore formation with sinapaldehyde. For this experiment, we chose to analyze alanine, arginine, aspartic acid, glutamic acid, and leucine because they are reported to be the five most relatively abundant amino acids present in plants.[57] We hypothesized that out of these five amino acids, arginine would most likely react with sinapaldehyde because, like proline, it contains secondary amine groups.

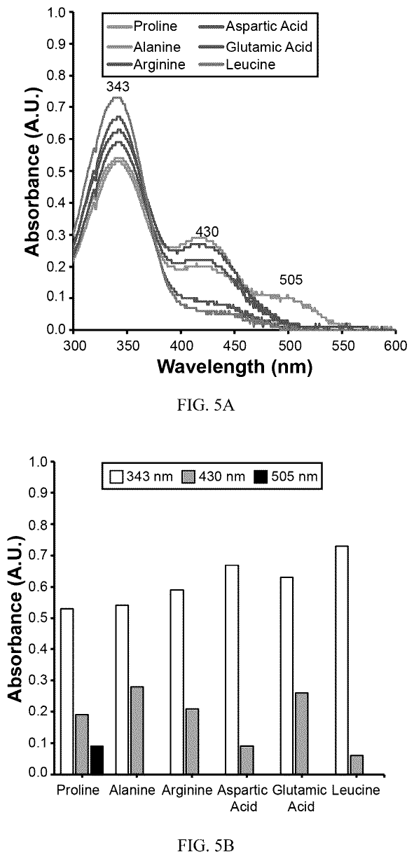

We combined sinapaldehyde, NaOH, and each amino acid at a 1:1:1 molar ratio in an aqueous environment and compared each absorbance profile to that of nesocodin. We observed no peak formation at 505 nm for the non-proline samples (FIGS. 5A-B) indicating that there is no chromophore formation that could interfere with the nesocodin signal. We also noticed that there was minimal sinapaldehyde deprotonation (430 nm peak) in the aspartic acid and leucine samples compared to the other conditions. To further illustrate the specificity of the sinapaldehyde-proline reaction, we increased the rate of the reaction by increasing the ratio of sinapaldehyde to amino acid to a 1:25 molar ratio. Our previous results highlighted that the 1:25 molar ratio of sinapaldehyde to proline led to the greatest nesocodin formation (FIGS. 4A and 4B). Under these conditions, the peak intensity at 505 nm in the proline sample was approximately seven times greater than the peak intensities of the other samples (FIGS. 4C and 4D). The non-proline samples had minimal peak formation at 505 nm, which suggests that there may be some formation of a sinapaldehyde-amino acid complex, but no prominent color formation compared to proline. Additionally, there was minimal peak formation at 505 nm in the arginine sample, which further supports that color formation is favored in the reaction between sinapaldehyde and proline.

### Development of Paper-Based Proline Sensors

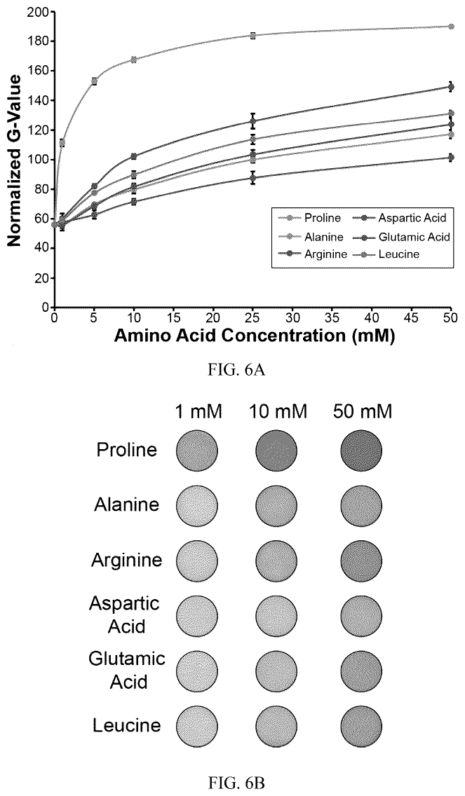

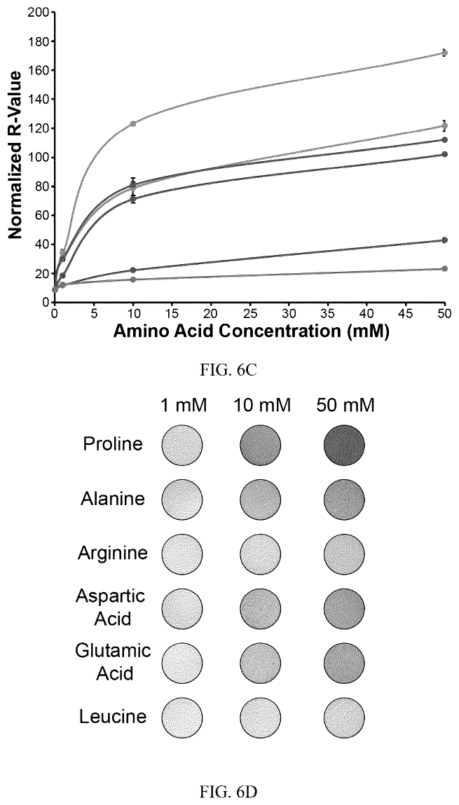

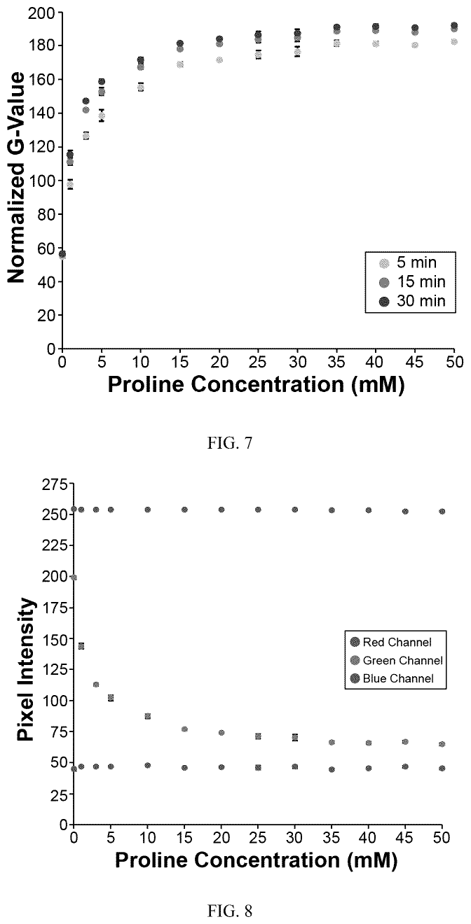

To translate this detection chemistry into a deployable device format, we created simple sensors by embedding sinapaldehyde in chromatography paper. We elected to use WHATMAN® 5 paper as the sensor substrate to evaluate whether the small pore size (2.5 μm) would aid in localizing the reaction between the small molecule detection agent, sinapaldehyde, and the analyte, proline, to produce a uniform color distribution. We applied solutions of proline prepared in water at increasing concentrations (0, 1, 5, 10, 25 and 50 mM) to the paper sensor to determine if we could initiate nesocodin formation when the reagents are combined in a porous matrix (FIGS. 6A and 6B). We first established that we achieve the most uniform color in our sensors when a full planar face of the sensor makes conformal contact with a volume of sample. Applying the analyte to the center or side of the sensor (i.e., from a defined introduction zone) led to the formation of a dark red ring around the edge of the paper disc following the flow path of the sample fluid. Additionally, we tested the length of time needed for the color to fully develop in the sensor and discerned that there was no consequential change in sensor color after a 15-minute reaction time (FIG. 7).

With a wide range of proline concentrations associated with different sample types, stress events, and sample processing protocols in the literature,[22a, 25a, 27a, 28-29, 41, 58] we aimed to identify an analyte concentration range for characterization of our sensors that would translate directly to analysis of real samples. Because proline concentrations from plant samples vary based on the mass of sampled plant material and volume of extraction solvent in each experimental protocol, we converted literature-reported quantities of proline in plant samples, reported in mg proline per gram of plant tissue, to molarity based on material quantities that could support analyte extraction in the field with minimal equipment-0.5 g of sampled plant tissue per 1 mL of extraction liquid. In Table 2, we report estimated proline concentrations from real plant samples that vary based on experimental conditions, but nearly all fall within the detection range of our sensors.

TABLE 2
Tabulated proline concentrations in
millimolar from literature results.

| | Reported Proline | Reported Proline |
|---|---|---|
| | Concentration | Concentration |
| | (No Stress)* | (Stress)** |
| Plant | (mM) | (mM) |
| Malva parviflora L.^{[6]} | 3.0 | 12.5 |
| Plantago major L.^{[6]} | 2.8 | 13.5 |
| Rumex vesicarius L. ^{[6]} | 2.8 | 10.1 |
| Sisymbrium erysimoides^{[6]} | 1.9 | 22.5 |
| Phaseolus vulgaris L.^{[7]} | ~0.6 | ~8.0 |
| Lycopersicon esculentum L.^{[8]} | 1.1 | 2.9 |
| Vitis vinifera L.^{[9]} | 133.1 | 341.7 |
| Zea mays L. cv. Saccharata^{[10]} | 81.8 | 300.5 |
| Zea mays L. cv. Ceratina^{[10]} | 59.3 | 170.0 |
| Oryza sativa^{[11]} | ~0.8 | ~5.2 |
| Brassica juncea^{[11]} | ~1.7 | ~5.2 |
| Vigna radiata^{[11]} | ~1.0 | ~3.0 |
| Capsicum annuum L.^{[12]} | ~0.07 | ~0.3 |
| Arabidopsis^{[13]} | ~0.5 | ~25 |
| Quercus robur L.^{[14]} | 32.5 | 139.8 |
| Cicer arietinum L.^{[15]} | ~2.2 | ~4.3 |
| Zea mays L. cv. RX947^{[16]} | 0.4 | 2.9 |
| Glycin max L.^{[17]} | ~0.5 | ~9.0 |
| Medicago sativa L. cv. Defi^{[18]} | 0.04 | 0.4 |

*Plant samples were controls in the article and were exposed to no environmental stress
**The highest reported concentration of proline in the plant sample after exposure to environmental stress.

Values with a ˜ symbol indicate that the value was approximated from a graph while the other values were reported in a table.

While there are some unique cases describing proline concentrations that could not be measured by our sensors (e.g., 1 M),[59] we expect that our measurement protocol will be sufficient for many plant species and stress events, but could be modified (e.g., sample dilution) to accommodate expanded measurement requirements.

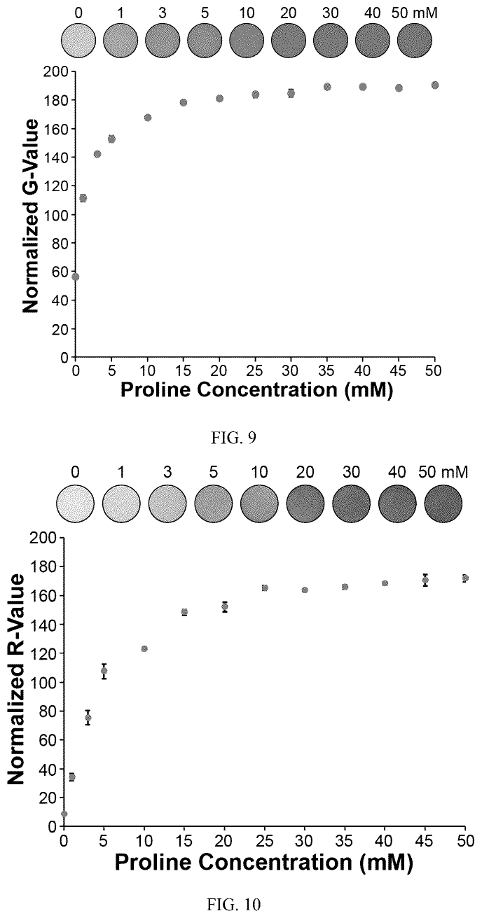

Based on our assessment of proline concentrations in a variety of plant species and stress conditions, we selected a biologically-relevant analyte concentration range for characterizing the performance of our sensors. We observed colorimetric responses dependent on proline concentrations in our sensors (FIG. 6A). Control samples containing no proline provided yellow signals and as proline concentrations increased, the sensors shifted to a light orange color at low proline concentrations, to dark orange, and then to red at the highest proline concentrations (FIG. 6B). We analyzed sensor color intensities using the RGB color space, isolating each of these color channels in ImageJ to measure changes in pixel intensity with increasing proline concentrations, and found that the green channel (G-value) intensity was the most sensitive to the color changes created in our devices (FIG. 8). To identify the useful measurement range of these sensors, we increased the concentration increment between the 0 and 50 mM range of our calibration curve (FIG. 9). Our sensors were most sensitive to changes in proline concentration ranging from 0 to 10 mM, and then the signal intensity began to plateau starting at approximately 15 mM. Across the 0 to 10 mM sample concentration range, the pixel intensity increased by 112 units on average, whereas pixel intensity only increased by 12 units on average across 15 mM to 50 mM proline samples. Overall, these results highlight that the analytical performance of our sensors is best within the 0-10 mM range, and that measurements of higher proline concentrations may be best suited for qualitative assessments of plant health. The concentration-dependent color changes of our sensors can be interpreted by eye to qualitatively differentiate between unstressed and stressed samples, and could be supported by accessories (e.g., color read guides)[60] or portable color analysis approaches (e.g., mobile image analysis applications) [61] in the future to enable semi-quantitative or quantitative measurements on-location.[60]

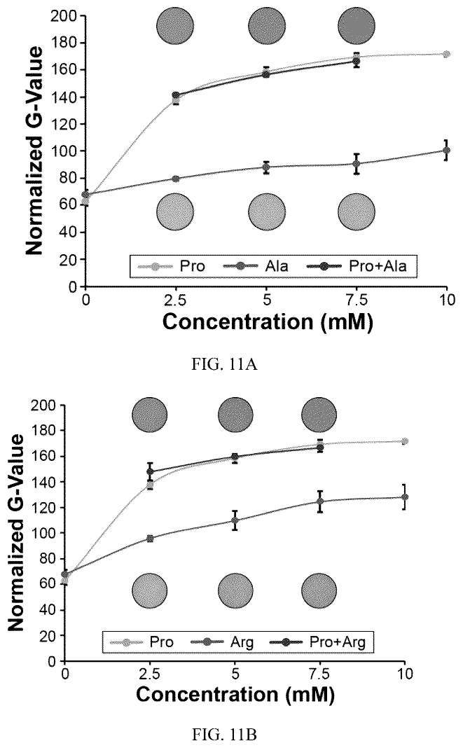

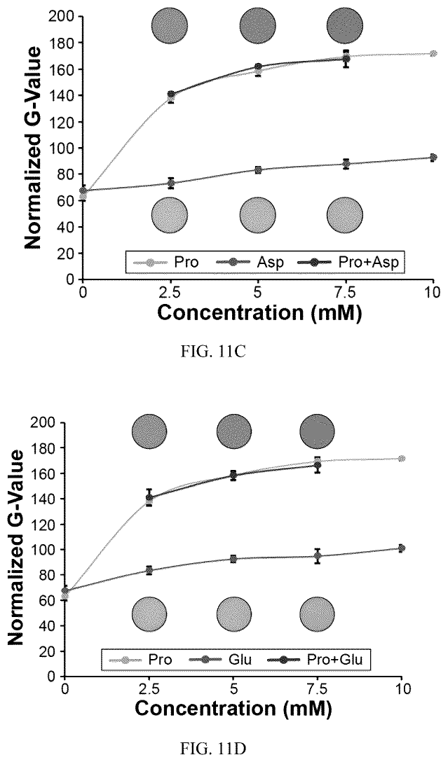

To directly compare the performance of our sinapaldehyde-based sensors to a standard approach for colorimetric quantification of proline, we created paper-based sensors using isatin, which reacts with proline and hydroxyproline to form a blue-colored product upon heating.[40b, 44] We evaluated these sensors using experimental parameters including activation temperature and sample pH demonstrated in the characterization of isatin-based proline sensors,[42] and heated them (6.5 minutes at 120° C.) to achieve uniform development of the colorimetric product. The isatin sensors achieved a greater overall color change in a single RGB channel (R-value) for 0 and 50 mM proline samples than our sinapaldehyde-based sensors, where the total change in pixel intensity was 134 and 163 units on average for sinapaldehyde-based sensors and isatin-based sensors, respectively. However, this difference may be due in part to how well the accessible color range of each sensor is captured within a particular color channel. Isatin-based sensors provided similar analytical performance to our sinapaldehyde-based sensors, where we observed high analyte sensitivity in the 0-10 mM range followed by signal saturation (FIGS. 2C and 10). Perhaps the greatest difference between the two reactions was that the color of the isatin-based sensors began to saturate and plateau at approximately 25 mM instead of 15 mM (FIG. 10). These results indicate that the colorimetric formation of nesocodin resulting from the room-temperature reaction between sinapaldehyde and the target analyte offers comparable analytical performance to established colorimetric strategies for measuring proline.

### Interfering Amino Acids in Paper-Based Sensors

Next, we evaluated whether other amino acids could react with sinapaldehyde to produce colorimetric responses in our paper-based sensing format. While we observed minimal interaction between sinapaldehyde and the amino acids alanine, arginine, aspartic acid, glutamic acid, and leucine in an aqueous solution (FIGS. 4C-D), confirmation that non-specific color formation would not confound proline detection was a critical step in the characterization of our sensors. We first observed that there was minimal color change when we added 1 mM of these amino acids to the sensors compared to that of proline (FIGS. 6A and 6B). Additionally, none of the other amino acids at 50 mM produced the intense red color that we observed when high concentrations of proline were present. The colorimetric signal of each of the other amino acids at 50 mM was lower than the colorimetric signal of proline at 3 mM, indicating that the color change generated by the other amino acids was minimal compared to the color change from proline. Across all of the potentially interfering amino acids we tested, arginine created the most intense colorimetric response in our sinapaldehyde-based sensors, which matched the results of our spectrophotometric analysis in bulk solution. Overall, these results highlight that while sinapaldehyde-based sensors are responsive to the presence of other amino acids, the color change is significantly less prominent than the response from proline and can be differentiated from color produced by measurable quantities of proline in a sample.

Because we observed some signal development in our sinapaldehyde-based sensors with potentially interfering amino acid species, we evaluated the specificity of isatin-based sensors for proline. We tested amino acid concentrations of 1, 10, and 50 mM in isatin-based sensors and compared the resulting color intensities to those provided by proline (FIG. 6C). Following the same trend as the sinapaldehyde-based sensors, each amino acid caused the isatin-based sensors to change color, though the color change was not as prominent as it was with proline (FIG. 6D). The signal intensity from sensors treated with 10 mM proline was 123±1 units, an intensity approached by sensors treated with 50 mM alanine, aspartic acid, and glutamic acid. Interestingly, sensors treated with arginine and leucine at 50 mM turned light pink instead of the deep blue color usually generated by interactions between isatin and proline. Overall, these results confirm that this accepted approach to quantifying proline is susceptible to interference from other amino acids. In our sinapaldehyde-based sensors, the signal of each interfering amino acid at 50 mM was lower than the signal of proline at 3 mM (FIG. 6A), while in isatin-based sensors signals from 50 mM interfering amino acids were lower than the signal created by 10 mM proline. This suggested that the proline detection reaction in our sinapaldehyde-based sensors is less susceptible to interference from other amino acids than the detection reaction between proline and isatin. These results demonstrate that while specific detection of proline using small-molecule colorimetric indicators remains challenging, distinct signal intensities provided by sinapaldehyde-based sensors in response to the target analyte may provide a performance advantage over existing colorimetric detection strategies.

Recognizing the need for our sensors to be functional in highly complex sample matrices, we explored their performance using samples comprising both proline and potentially interfering amino acids. We chose to analyze samples containing additional amino acid concentrations 3 times that of proline based on a critical analysis[57] of more than 100 publications which concluded that glutamic acid is, on average, the most abundant amino acid in plant tissues at approximately 2.5 times the abundance of proline. By capturing this range of amino acid concentrations, we sought to evaluate the performance of our sensors against sample matrices that were broadly representative of typical plant chemistry. To understand what concentrations of interfering species to expect in samples of stressed plant tissue, we surveyed publications describing changes in the amino acid profiles of plants in response to controlled stresses. Our search revealed highly variable reported results based on the plant species under study, stress method, and differences in sample processing and analysis protocols.[24b, 62] However, the approximate concentrations of amino acids in stressed plant samples rarely exceeded the concentration range that we used to perform preliminary characterization of our sensors (Table 3).

TABLE 3
Changes in free amino acid concentrations from environmental stress

| | Reference 19^{[19]} | | Reference 20^{[20]} | | Reference 21^{[21]} | | Reference 22^{[22]} | |
|---|---|---|---|---|---|---|---|---|
| | Control | Stress | Control | Stress | Control | Stress | Control | Stress |
| Amino acid | (mM) | (mM) | (mM) | (mM) | (mM) | (mM) | (mM) | (mM) |
| Alanine | 9.4 | 28.2 | 1.0 | 0.5 | 10.7 | 6.6 | 16.4 | 9.5 |
| Arginine | | | 0.2 | 0.3 | ND | 0.9 | 7.6 | 7.7 |
| Asparagine | 0.4 | 13.0 | 0.8 | 1.0 | 4.7 | 31.3* | | |
| Aspartic Acid | 12.5 | 30.7 | 1.2 | 0.5 | 3.5 | 4.9 | 18.4 | 12.1 |
| Cysteine | | | 0.8 | 1.0 | ND | 0.3 | | |
| Glycine | 2.1 | 10.3 | 1.3 | 1.8 | 0.4 | 0.9 | 18.3 | 14.3 |
| Glutamine | 4.8 | 121.4 | 0.9 | 1.5 | | | | |
| Glutamic Acid | 25 | 19.3 | 1.9 | 0.7 | 11.15 | 2.7 | 12.0 | 9.6 |
| Histidine | 0.3 | 0.4 | 0.2 | 0.2 | ND | 0.7 | 4.3 | 5.0 |
| Isoleucine | 0.5 | 1.0 | | | ND | 0.8 | 9.7 | 8.9 |
| Leucine | 0.5 | 0.9 | 0.7 | 0.4 | | | 16.0 | 14.0 |
| Lysine | 0.4 | 0.5 | 0.2 | 0.2 | 0.2 | 0.7 | 12.8 | 12.6 |
| Methionine | 0.4 | 0.7 | 0.1 | 0.1 | | | 1.7 | 1.8 |
| Phenylalanine | 0.3 | 0.6 | 0.1 | 0.3 | | | 7.6 | 6.7 |
| Proline | 0.5 | 23.8 | 1.1 | 17.0 | 0.6 | 63.0 | 7.6 | 11.9 |
| Serine | 13.2 | 14.0 | 1.0 | 0.9 | 4.0 | 6.7 | 15.2 | 12.2 |
| Threonine | 2.6 | 4.2 | 0.8 | 0.9 | 4.7 | 31.3* | 10.3 | 8.8 |
| Tryptophan | 0.3 | 0.3 | | | | | | |
| Tyrosine | 0.4 | 0.6 | 0.1 | 0.2 | | | 3.7 | 3.4 |
| Valine | 2.6 | 6.4 | 0.5 | 0.7 | 0.7 | 4.2 | 11.4 | 12.3 |

*Indicates that the authors detected asparagine and threonine together
ND indicates not detected

Because the signal intensities provided by high concentrations of amino acids that could be abundant in stressed plant samples were more than 16 times lower than the signal intensities created by small quantities of proline in our sinapaldehyde-based sensors, we evaluated whether they would provide reliable quantification of proline from complex sample matrices. While the analytical performance of these sensors may not be suitable for every measurement scenario or sample composition, we designed our experiments to evaluate their utility for a broad range of sample types and environmental stresses.

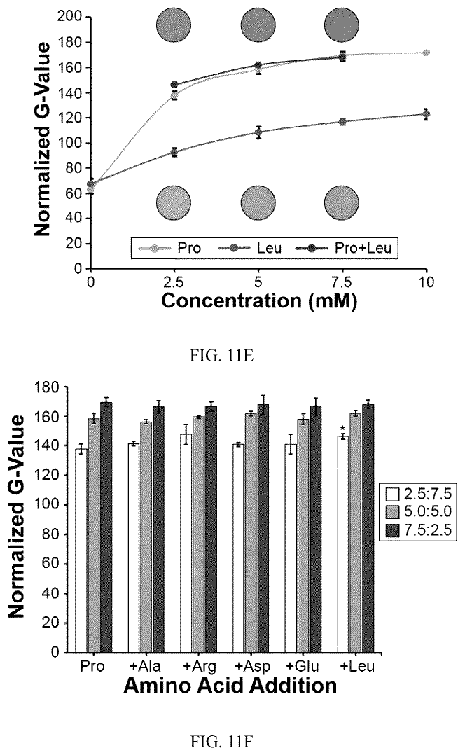

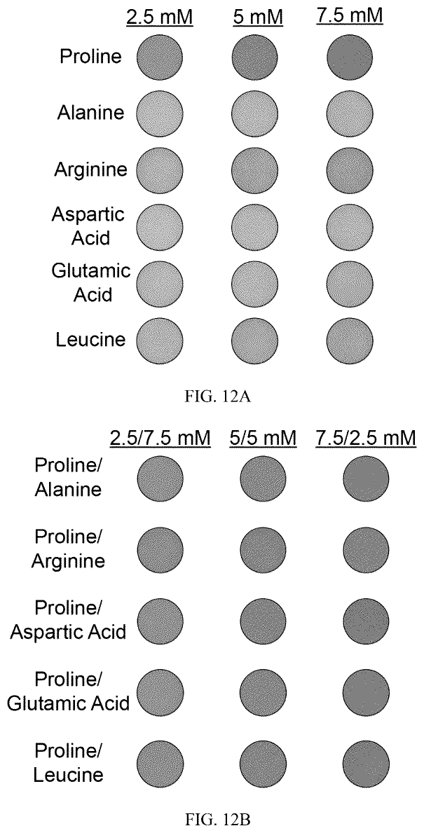

To confirm that signal from other amino acids did not preclude proline detection using sinapaldehyde-based sensors, we evaluated complex sample matrices comprised of 10 mM total amino acid content with different molar ratios (1:3, 1:1, and 3:1) of proline to an interfering species. [42] We also separately evaluated each interfering amino acid and proline at 2.5, 5, 7.5, and 10 mM sample concentrations. Our results revealed that for each combination of an interfering amino acid and proline, the colorimetric response from the sensors overlaps the signal from the sensors treated with pure proline (FIGS. 11A-E and FIGS. 12A-B). For example, samples of proline and arginine generated pixel intensities of 148±7 units, 160±1 units, and 167±3 units for 1:3, 1:1, and 3:1 molar ratios of proline to arginine, respectively, while pure proline samples produced intensities of 138±3 units, 158±4 units, and 169±3 units for 2.5 mM, 5 mM, and 7.5 mM, respectively. These results illustrate that the arginine was neither amplifying nor diminishing color development in the sensor in the presence of proline. Generally, there was no statistical difference between the colorimetric responses from sensors treated with complex samples (i.e., proline and another amino acid) and pure proline (FIG. 11F). The only exception was for proline and excess leucine in a 1:3 molar ratio, but equimolar and proline-dominant samples provided signals that correlated closely with those provided by proline controls. We observed that the colorimetric responses of proline-containing samples approximated the signal intensities provided by proline alone, and that the interfering species alone provided much lower intensity signals that could be visually distinguished from those provided by proline-containing samples (FIGS. 11A-F). These results indicate that across the effective concentration range of our sinapaldehyde-based sensors, the presence of additional amino acids does not augment or mitigate colorimetric signal formation from the target analyte. In other words, the signal produced by proline and other amino acids in a complex sample is not additive and will not interfere with quantitative measurements of proline.

### Proline Detection in Plant Samples

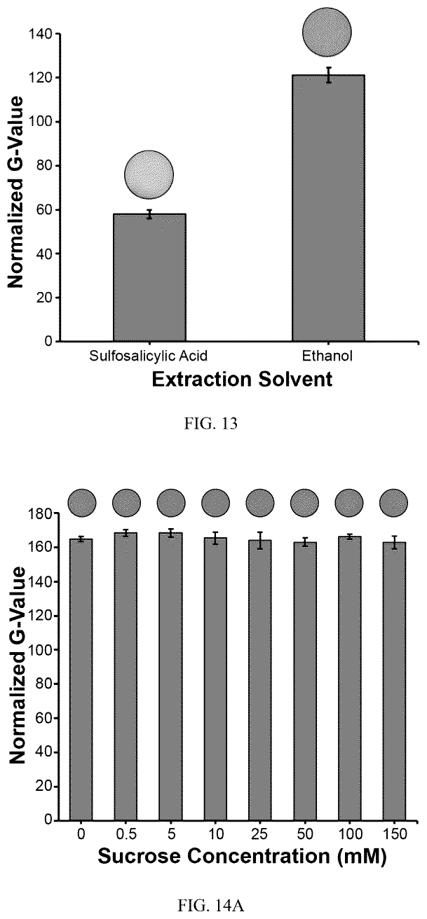

Our next goal was to demonstrate the utility of our sensors by detecting proline from actual plant samples. First, we needed to identify a liquid matrix to extract proline from plant tissue and evaluate its compatibility with our sensors. We decided to test sulfosalicylic acid and ethanol because both have been reported as effective solvents for proline extraction.[40a, 54] To explore extraction efficiency and compatibility with our colorimetric detection scheme, we ground ornamental cabbage leaves and added them to each matrix at a ratio of 0.5 g of plant per 1 mL of solvent. We performed these extractions at ambient temperature to eliminate the need for secondary electronic equipment and model an experimental protocol that would be compatible with on-site testing. We supplemented sulfosalicylic acid samples with 250 mM NaOH to ensure that the pH of the solution was basic to support nesocodin formation, while ethanol samples contained 50 mM NaOH to match the conditions we used to develop the sinapaldehyde-based sensors. We treated our sinapaldehyde-based sensors with each extract and observed that the color changes in samples treated with ethanol was approximately double that of the sensors treated with sulfosalicylic acid samples (FIG. 13). These results suggested that either the ethanol provided a more effective extraction than sulfosalicylic acid, or that extraction with sulfosalicylic acid released an interfering species that attenuated nesocodin formation.

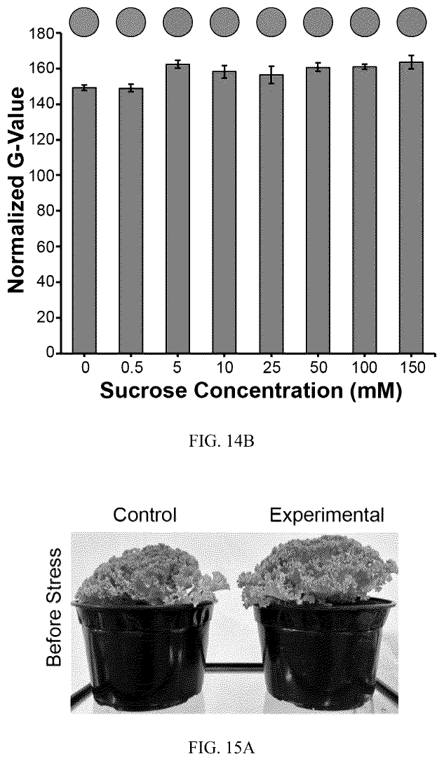

Sugars that can be solubilized with proline in aqueous extraction matrices have previously been reported to interfere with other colorimetric proline assays. [43, 51] We hypothesized that the sulfosalicylic acid may extract sugars from plant leaves with proline, resulting in inhibition of the sinapaldehyde-proline colorimetric reaction. To test this, we prepared 10 mM proline samples in both water and 3% sulfosalicylic acid with increasing concentrations (0 to 150 mM) of sucrose, based on a wide range of reported sugar concentrations in the Brassica genus. [63] However, our results for both conditions showed little change in the colorimetric response achieved by 10 mM proline in our sinapaldehyde sensors (FIGS. 14A-B). In fact, higher concentrations of sucrose appeared to enhance the color of the sensors in sulfosalicylic acid samples (FIG. 14B). We selected ethanol as the extraction matrix for our case studies.

For our first experiment, we demonstrated that we could use our sensors to qualitatively identify increased proline concentrations in plants resulting from controlled stress. We purchased two identical ornamental cabbage plants (FIG. 15A) and created osmotic stress in one plant by watering it with a salt solution incrementally over the course of 12 hours. We imaged both plants after this treatment to highlight that there were no visible signs of duress after the stress event (FIG. 15B). Afterwards, we removed leaves from both plants, where the control plant was watered with tap water and processed them for analysis. One portion of the sampled leaves was frozen for future analysis, while the other was immediately dried according to established protocols for proline measurement.[22b] We also collected samples before inducing osmotic stress in the experimental plant to have reference measurements for both subjects. The yellow color of sensors that measured unstressed samples indicated that there was no detectable proline in either plant before the stress event (FIG. 15C). Both samples of frozen leaf generated a colorimetric response that indicated the presence of proline. The experimental plant had a greater increase in signal than the control plant compared to our pre-stress reference measurements. Signal intensities increased by 46 units and 84 units on average for the control and experimental plants, respectively (FIG. 15C). While we did not expect to observe increased proline production by the unstressed control plant, the process of clipping leaves from the plant and subjecting that tissue to freezing temperatures may have led to a protective response resulting in proline accumulation. However, these results demonstrated that our sinapaldehyde-based sensors can effectively measure proline from real samples to identify stressed vs. unstressed plants before visible stress indicators arise.

For the dried leaf samples, extracts from the control and experimental plants provided comparable colorimetric responses in the sensors, with pixel intensities of 176±1 units and 178±2 units for the control and experimental samples, respectively (FIG. 15C). Although both the frozen and dried samples were collected from the stressed and unstressed plants at the same time, the process of dehydrating the dried samples in preparation for analysis took considerably longer than the freezing step (i.e., days vs. hours) and concentrated the proline within the dry plant tissue, leading to much higher signal intensities for dried vs. frozen samples. While the sampling and freezing process likely induced some additional and unintentional stress in our cabbage samples, this approach still enabled differentiation of stressed and unstressed plants. However, drying samples in preparation for analysis led to so much proline accumulation that it was not possible to differentiate stressed vs. unstressed plants. These differences are supported by our statistical analysis (FIG. 15C), where the frozen control and experimental plants demonstrated significantly different responses, while the dried samples provided responses that were not statistically different. These results highlight how sensitive plants can be to established sample processing protocols and conditions, indicating that a faster, more direct extraction approach immediately after leaf sampling may more accurately capture the health status of plants using our sinapaldehyde-based sensors.

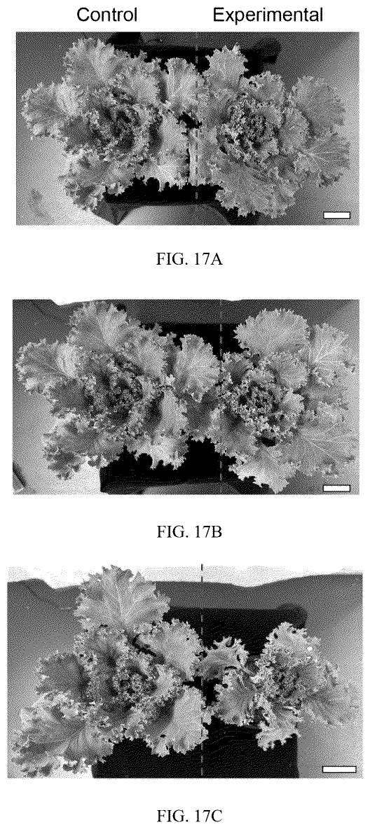

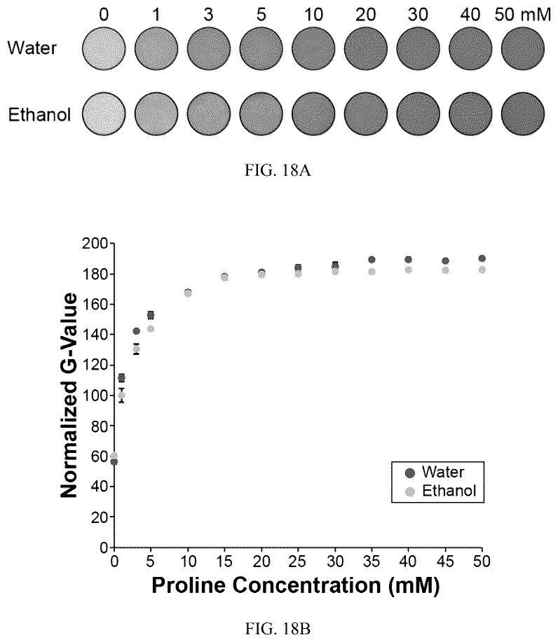

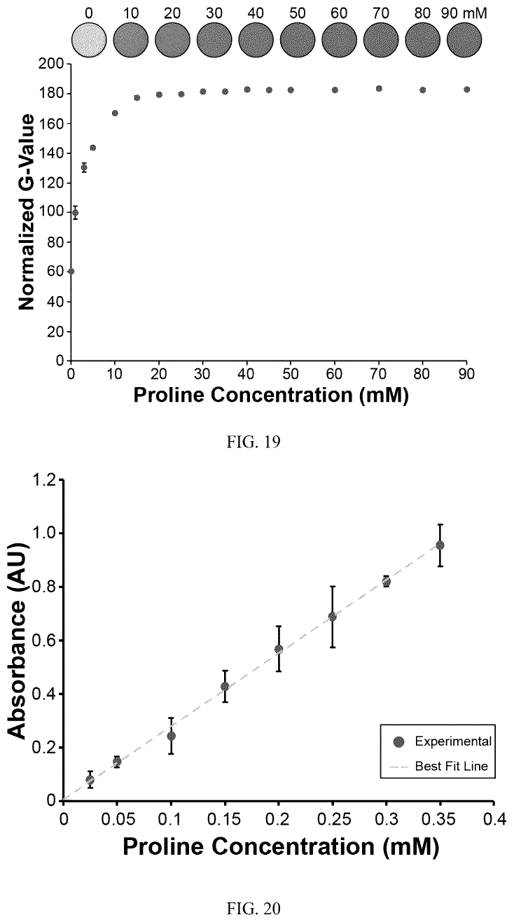

Next, we evaluated whether our sensors could be used to quantitatively determine proline concentrations in plant tissue. For these experiments, we selected ornamental kale plants and applied two thermal stress cycles (35° C. for 24 hours) (FIGS. 16A and 17) to the experimental plant to demonstrate the versatility of our approach. After each stress event, we clipped leaf samples, extracted proline using ethanol, and performed colorimetric measurements using our sinapaldehyde-based sensors. The sampling and analysis were completed within 30 min. of the conclusion of each stress event to model rapid, on-site measurements using our sinapaldehyde-based sensors and limit induction of stress and proline accumulation by the sampling and sample preparation process. To connect colorimetric signals to numerical concentrations of proline in our extract samples, we prepared a calibration curve using known concentrations prepared in basic ethanol. Overall, colorimetric responses to increasing proline concentrations in ethanol followed the same trend and color progression (FIGS. 16B and 18) that we observed in aqueous conditions. While there are small differences between maximum signal intensities at our highest proline concentrations, which may be due to minor inconsistencies in sinapaldehyde loading during the sensor fabrication process, the calibration curves consistently plateau at 15 mM proline (FIG. 18B). We expanded the analyte concentration range to 90 mM proline to confirm that signals remained saturated with increasing analyte concentrations (FIG. 19).

Using a modified four-parameter logistic curve fitting equation (Equation 2 and 3), we calculated the proline concentrations of real plant samples using our calibration curve (Equation 4 and FIG. 16B).

$$y = d + \frac{a - d}{1 + {( \frac{x}{c} )}^{b}} \qquad Equation 2$$

$${y}_{norm} = {y}_{0} - {y}_{x} \qquad Equation 3$$

$$y = 12 \; 7.1 + \frac{- 1 26.1}{1 + {( \frac{x}{2.4} )}^{1.1}} \qquad Equation 4$$

After the first thermal stress event, the control kale plant contained about 1.8±0.2 mM proline and the experimental plant contained 2.9±0.2 mM proline (FIG. 16C). After the second stress event, proline concentrations increased to 7.3±1.1 mM in the control plant and 14.3±2.6 mM in the experimental plant (FIG. 16C). For each stress event, the proline concentration of the experimental plant was greater on average and statistically different from the proline concentrations of the controls. While we did not expect to observe proline accumulation in the control plant, removal of leaves for measurements performed after the first heating cycle may have resulted in unintentional induced stress, indicating that the size of a plant relative to the size of sampled leaves should be taken into consideration for repeat measurement events. Overall, these results highlight that our sinapaldehyde sensors can support rapid, on-site measurements of plant health without requirements for centralized processing steps such as heating, freezing, or drying, and measure environmental stresses in plants before the onset of visually identifiable symptoms.

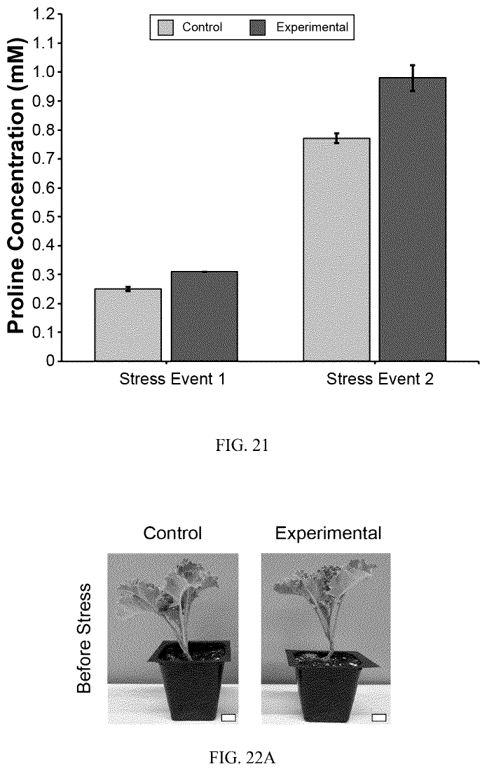

To compare the results provided by our sinapaldehyde-based sensors to an established plant stress test protocol, we performed an acid ninhydrin assay in parallel to our colorimetric measurements of thermally stressed ornamental kale. Following a standard protocol, [40a] we prepared our standards for the calibration curve (FIG. 20) and extracted proline from the kale plants using 3% (w/v) aqueous sulfosalicylic acid. We chose to extract the proline with sulfosalicylic acid for the ninhydrin assay as opposed to ethanol because we observed poor phase separation between ethanol and toluene during the ninhydrin assay, which is required for isolation of the chromophore that is measured by spectrophotometry in this approach. The extraction time and temperature were the same for both the ethanol and sulfosalicylic acid extractions used for our colorimetric sensor characterization and the acid ninhydrin assay, respectively. We determined that the control and experimental plants had proline concentrations of 0.25±0.01 mM and 0.31±0.00 mM after the first stress event and 0.77±0.02 mM and 0.98±0.04 mM after the second stress event (FIG. 21). We suspect that differences in proline extraction efficiency and sample processing steps between these parallel experiments led to differences between measurements performed using these two methods. However, the results of the acid ninhydrin assay correlated well with measurements performed using our sinapaldehyde-based sensors-we observed that after the first stress event the experimental plant had a higher concentration of proline than the control plant and that the concentration of proline in both plants increases after the second stress event, again with the concentration of proline in the experimental plant being higher than that of the control. These results highlight that our sinapaldehyde-based sensors are a reliable and effective tool for detecting and measuring proline concentrations in real plant samples, despite differences from established methods that may result from differences in extraction efficiency or sample processing.

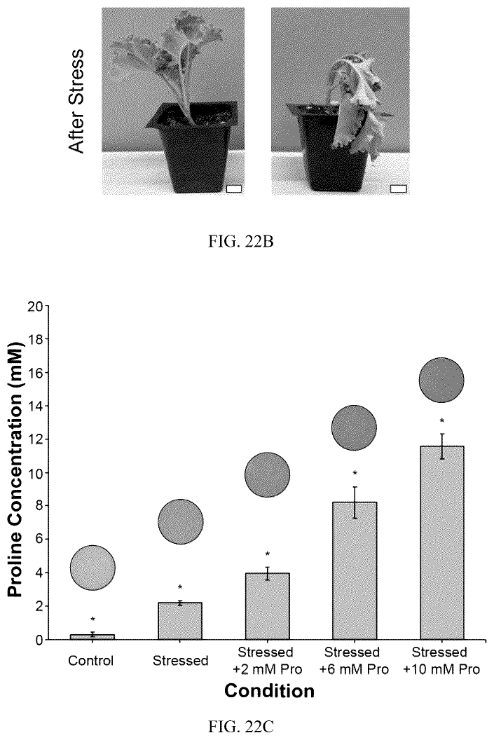

To demonstrate that our sensors could specifically detect proline within complex biological sample matrices, we performed a second thermal stress experiment using cabbage plants. The purpose of this experiment was to confirm that increasing color intensities in sensors measuring stressed plant tissues were due to corresponding increases in proline concentrations and not undesirable reactions with amine-containing components generated by stress or sample processing events. We supplemented extract from a stressed plant sample with 3 linearly increasing concentrations of exogenous proline to test whether the resulting changes in signal intensity would correspond directly to known increases in analyte content. In the same manner as the previous experiment with kale plants, we exposed an immature green cabbage plant (experimental) to 35° C., 40% humidity conditions for 24 hours, and kept another plant in ambient conditions as the control (FIGS. 22A and 22B). As we observed previously (FIG. 16), the stressed plant provided a greater colorimetric response than the control plant. We used our calibration curve (FIG. 16B) to determine that the proline concentrations in the control and experimental plant were 0.3±0.1 mM and 2.2±0.2 mM, respectively (FIG. 22C). Next, we added exogenous proline to the extract of the stressed plant so the final concentration of proline in the sample was the aggregate of the original proline content of the extract and an additional 2, 6, or 10 mM proline. We measured these samples using our sinapaldehyde-based sensors and found that proline concentrations increased from 2.2±0.2 mM to 4.0±0.4 mM, 8.2±0.9 mM and 11.6±0.8 mM for the 2 mM, 6 mM, and 10 mM proline additions, respectively, and each provided a statistically significant color change from each of the other evaluated conditions (FIG. 22C). Overall, these results reinforce that the color of nesocodin, which is formed only by the reaction between sinapaldehyde and proline, is unique to the target analyte and enables quantification of proline without interference from competing reactive molecules in complex biological sample matrices.

### Device Design and Fabrication

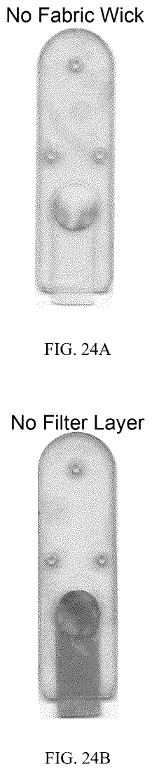

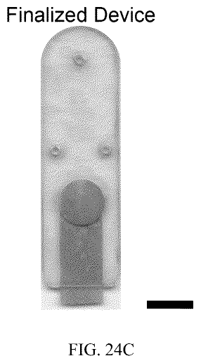

Next, we incorporated our sensors into an autonomous microfluidic device to demonstrate their potential as a solution for use at the point of need. Our goal was to eliminate requirements for user intervention and liquid handling steps by designing a device that could uniformly deliver sample from the plant extract mixture to the sinapaldehyde-based sensor. We developed an additively manufactured microfluidic system, fabricated by lamination of pin-aligned plastic layers backed with pressure-sensitive adhesive, that capture the sensor against a high-flow wicking material that transports the sample by capillary action (FIG. 23A). In our initial efforts to deliver sample fluid to the sensor using porous matrices, we observed that chromatography paper could not drive flow quickly enough to uniformly saturate the sensor, which lead to heterogenous rehydration and transport of sinapaldehyde during nesocodin formation (FIG. 24). To create a uniform distribution of signal within the device, we sought a high-flow material that could quickly distribute fluid beneath the full surface area of the sensor to enable uniformly distributed rehydration of sinapaldehyde and formation of nesocodin. We found that a commercial absorbent sham cloth (FIG. 23B) provided rapid fluid flow to the sensor but filled the sensor inconsistently (FIG. 24). We hypothesized that this was because the large pore size of the sham cloth led to inconsistent contact with the bottom surface of the sensor. To address this challenge, we added a disc of chromatography paper with the same dimensions as the sensor between the sensor and sham cloth. We selected a different grade of chromatography paper (WHATMAN® 3) because it has a larger pore size (6 μm) than the chromatography paper we used to fabricate our sensors (WHATMAN® 5, 2.5 μm) which gradually tightened the capillary matrix in the direction of flow through the device, resulting in uniform distribution of sample fluid and analyte within these devices. This strategy provided uniform signal formation across the useful analyte concentration range of our devices (FIG. 23C), enabling interpretation by visual inspection or image analysis.

As a final proof-of-concept experiment, we used these devices to detect changes in the proline concentration of a biological sample. To test our devices, we used extract from green cabbage plants subjected to 24 h of thermal stress or storage in ambient conditions. Interestingly, extracts prepared with 50 mM NaOH to facilitate nesocodin formation created no color change within the sensor embedded within the device. We suspected that the typical concentration of NaOH that supported color development during characterization of our sensors may have been depleted by interactions with components of the sample matrix during fluid transport to the sensor along the tortuous path of the sham cloth. When we increased the concentration of NaOH in the sample matrix to 250 mM, we observed color changes from samples extracted from unstressed and stressed plants (FIG. 23D). In summary, our device design enables dry storage and protection of functional sensing components, transports sample fluid from the extraction container to the sensor within 1 minute, and enables a simplified, user-friendly measurement protocol for biological samples with no requirements for laboratory resources or equipment.

### CONCLUSIONS

Disclosed herein, in some embodiments, is a new paper-based sensor for quantifying proline in plants that leverages the reaction between sinapaldehyde and proline to form the natural red pigment nesocodin. To control nesocodin formation based on the molar ratio of sinapaldehyde to proline, we first evaluated the variables that contributed to nesocodin synthesis. Next, we embedded sinapaldehyde into chromatography paper to create color-changing sensors that shifted from yellow to dark red in response to increasing proline concentrations and were not inhibited by the presence of other amino acids or the components of biological samples. We tested our sensors against two different types of plants that were exposed to either osmotic or thermal stress, highlighting that these sensors can detect and quantify proline in real samples. Finally, we designed and developed a user-friendly device that enables on-site detection of plant stress without requirements for additional equipment. In some embodiments, this device supports qualitative assessments of plant health (i.e., healthy vs. stressed) by visual inspection, and it can also be paired with companion tools for more quantitative measurements. Furthermore, additional device features such as sample pH or internal reference or calibration components could expand the density of diagnostic information measured from a single plant sample, providing users with access to information required to make critical decisions that ultimately inform strategic interventions to improve agricultural yields and farming efficiency.

### Supporting Information

Nesocodin Synthesis in Methanol with Different Bases

We first prepared nesocodin in methanol by scaling down the protocol designed by Roy et al.[56] In summary, we dissolved 22.4 mg of sinapaldehyde into 1 mL of methanol for a final concentration of 0.109 M. We then added an equimolar amount of proline powder directly into the solution while stirring and let the proline completely dissolve. Next, we added 10 μL of tributylamine (2.6:1 molar ratio of sinapaldehyde to tributylamine) dropwise to the solution and left the reaction to stir for 15 minutes.

Next, we decided to test if we could synthesize nesocodin using sodium hydroxide (NaOH) instead of tributylamine. We prepared a 10 M NaOH solution in water so we would add minimal water to the methanol-based solution. We repeated the same protocol described above, except we used NaOH (2.6:1 molar ratio) in place of tributylamine. Additionally, we carried out this protocol with a 1:1 molar ratio of sinapaldehyde to NaOH. We let all reactions occur for 15 minutes.

After 15 minutes of reaction time, we measured the absorbance profile of each solution using an Ocean Optics spectrophotometer (SpectraMax M5 Series, Molecular Devices). We diluted each sample with methanol until the detector was not oversaturated and took absorbance profiles from 300 to 700 nm for each condition.

### Extinction Coefficient Measurements of Nesocodin in Methanol

To calculate the extinction coefficient of synthetic nesocodin prepared in methanol, we first followed the nesocodin synthesis protocol using NaOH that we previously described herein. For the analysis, we prepared diluted samples at six different concentrations from this initial solution and analyzed the absorbance profile of each sample from 300-700 nm with an Ocean Optics Spectrophotometer. To calculate the extinction coefficient, we used the Beer-Lambert Equation (Equation 1). In Equation 1, A is the absorbance at 520 nm, F is the extinction coefficient, l is the path length (1 cm) and c is the concentration of the analyte. We plotted the absorbance peak intensity at 520 nm against the concentration of nesocodin, and then used the slope of the best-fit line to determine the extinction coefficient of the nesocodin solution at 520 nm. The results are an average of three replicates, where the initial nesocodin solution was prepared three separate times, and the error of the extinction coefficient is the standard deviation.

### Extinction Coefficient Measurements of Nesocodin in Water

To calculate the extinction coefficient of synthetic nesocodin in water, we first prepared 0.005 M sinapaldehyde and 0.1 M proline in water. We then prepared a 2 mL solution consisting of 0.002 M sinapaldehyde, proline, and NaOH (1:1:1 molar ratio) in water, vortexed the sample, and let it sit for 15 minutes so the reaction could occur. Afterwards, we prepared six different diluted samples, each with a different nesocodin concentration, and measured the absorbance profile of the solutions on an Ocean Optics spectrophotometer. We followed the same steps for calculating the extinction coefficient as previously described (Equation 1), except we used the peak intensity at 505 nm. The results are an average of three replicates and error is reported as the standard deviation.

### Preparation of Amino Acid Solutions

We prepared 0.1 M solutions of alanine, arginine, aspartic acid, glutamic acid, leucine, and proline in deionized water. We dissolved the amino acids in 90% of the final required volume of water and then used 1 M hydrochloric acid and 1 M sodium hydroxide to adjust the pH of each solution to 7.2±0.1. We then brought the solution to its final volume and concentration with water.

### Synthesis of Synthetic Nesocodin and Evaluation of Sinapaldehyde Reactivity

We first prepared a 5 mM stock solution of sinapaldehyde in water. We adjusted the sinapaldehyde concentration in our experiments with water to 5 mM as opposed to 50 mM in the methanol experiments due to differences in solubility. Next, we mixed the sinapaldehyde stock with 0.1 M proline at increasing molar ratios of sinapaldehyde to proline (1:1, 1:5, 1:10, 1:15, 1:20, 1:25) where we kept the quantity of sinapaldehyde constant. We then added 0.1 M sodium hydroxide at a 1:1 molar ratio with the sinapaldehyde and brought each solution to a constant volume with water. We vortexed the samples and left them to react for 15 minutes.

To determine if there could be any chromophore formation with amino acids other than proline, we performed the same experiments described above, with alanine, arginine, aspartic acid, glutamic acid, or leucine substituted for proline.

For spectrophotometric analysis for all amino acids, we first added 70 μL of the sample to 3.93 mL of water to dilute the sample and prevent oversaturation of the detector. We transferred the diluted sample to a polystyrene cuvette and measured the absorbance profile of the sample from 300-700 nm using an Ocean Optics Spectrophotometer. We then identified the absorbance values of the peaks at 343, 430, and 505 nm.

### Preparation of Sinapaldehyde-Based Paper Sensors

To prepare our paper-based sensors, we first cut out 9 mm discs from WHATMAN® 5 filter paper using a craft punch. We chose WHATMAN® 5 paper because its small pore size (<2.5 μm) enabled localized rehydration of our small-molecule sensing agent, which provided uniform color intensity upon reaction with proline. We then prepared a 50 mM solution of sinapaldehyde in acetone and deposited 5 μL of the solution into each disc which filled the capillary volume of the paper without oversaturation, resulting in uniform deposition of sinapaldehyde. We suspended the sensor substrates over the holes in a pipette tip rack to ensure that the sinapaldehyde solution was not transferred to an underlying substrate. We then placed the sensors in a 60° C. oven for 10 minutes to remove the solvent.

### Calibration Curves for Sinapaldehyde-Based Sensors

We prepared two calibration curves using our sensors: one with sample solutions prepared in water and one with sample solutions prepared in 100% ethanol. We prepared 1 mL analyte solutions that contained increasing concentrations of proline (0, 1, 3, 5, 10, 15, 20, 25, 30, 35, 40, 45, 50 mM) and 50 mM sodium hydroxide. For the samples prepared in water, we introduced these analytes to our sensors by pipetting 6 μL droplets (required volume to fill but not oversaturate the sensors) of the analyte directly onto a flatbed photo scanner (Epson Perfection V39) and then placed the sensor directly on top of the droplet. We then covered the sensors with a thin plastic film (polyethylene terephthalate) to minimize evaporation, allowed color to develop for 15 minutes, and then obtained high-resolution images (600 dpi) of the samples. For the samples prepared in ethanol, we introduced the analyte by pipetting 8 μL of the analyte onto glossy cardstock (Leneta) and placed the sensor directly on top of the droplet. We chose to apply the analyte to the cardstock so the sample could retain its shape and not spread before we added the sensors. We then placed a glass microscope slide over the sensors for 5 minutes to prevent solvent evaporation during the reaction. Afterwards, we removed the slide and allowed the color to develop for an additional 15 minutes. We transferred the samples onto the flatbed scanner, flipping them in the process so the top of the sensor was now against the scanner surface, and obtained high-resolution images. Additionally, we tested samples with 60, 70, 80, and 90 mM proline prepared in ethanol.

To quantify the color of the sensors, we measured the pixel intensity in the RGB (red, green, blue) color space using ImageJ. We normalized the values in the green channel (G-values) of the RGB color space by subtracting each measured value from 255 and plotted these results to create our calibration curves. We performed three replicate measurements for each proline concentration. We reported the average value for each measured concentration and presented error as standard deviation.

Development of Proline Calibration Curve with Isatin-Based Sensors

First, we prepared a 0.05 M solution of isatin in acetone, and then applied 5 μL of the solution onto 9 mm discs of WHATMAN® 5 paper. We let the samples dry in a 120° C. oven for 10 minutes. Next, we prepared 0.1 M proline in 0.1 M MES buffer (pH=4.0) and then made aliquots of 0, 1, 3, 5, 10, 15, 20, 25, 30, 35, 40, 45, and 50 mM proline solutions. We then applied a 6 μL droplet of a proline solution onto a cleaned glass slide and then carefully placed the sensor on top of the droplet on the side where we applied the isatin. We placed the sensor in a way that the center of the sensor made contact with the droplet first, to ensure a uniform dispersion of the proline analyte across the paper. We allowed the samples to sit at room temperature for 3 minutes so the proline solution could fill the entire sensor. Then, we placed the sensors in a 120° C. oven for 6.5 minutes; the first 1.5 minutes of incubation was to allow the oven temperature to get back up to 120° C. after the door was opened, and the next 5 minutes were for the reaction to occur. After 6.5 minutes, we removed the samples from oven, placed another cleaned glass slide on top of the samples to fully flatten them, and scanned them on a photo scanner. We chose this temperature and sample pH for this experiment based on the results by Santhosh and Park who designed paper-based isatin sensors for proline detection.[42] We increased the reaction time of our sensors to produce a more uniform color across the sensor.

To quantify the color of the sensors, we used ImageJ to measure each channel in the RGB color space. Our results showed that color change was most prominent in the red channel. We normalized these values by subtracting the result from 255 and plotted the color in response to proline concentration. Each data point is an average of three sensor replicates and error is the standard deviation.

### Comparison of Proline Concentrations in the Literature

To confirm that the detection range of our sensors was relevant for analysis of real plant samples, we converted proline concentrations reported in the literature to molarity based on the extraction method we designed for preparation of biological samples. We chose values that were reported in milligrams of proline per gram of fresh plant tissue (mg/g) or in μmol proline per gram of fresh plant tissue (μmol/g) and first converted the mass of proline into moles. We decided that our extraction protocol would consist of extracting proline from plants at a ratio of 0.5 g of fresh plant tissue to 1 mL of extracting solvent. Using that ratio, we converted the mass of fresh plant tissue to correspond to a target volume of extract, which allowed us to express our calculated concentration values in molarity (M). This concentration was converted to millimolar (mM) for comparison to the detection range of our sensors. This approach assumes ideal extraction efficiency and does not account for the liquid content of plant samples in the calculation of the extraction volume. Our calculation steps can be seen below:

$$\begin{matrix}\frac{mg proline}{g fresh plant} \times \frac{1 g proline}{1000 mg proline} \times \frac{1 mol proline}{115.13 g proline} \times \frac{0.5 g fresh plant}{1 mL solvent} = \frac{mol proline}{mL solvent} = \frac{mol proline}{mL solvent} = mM of proline \\ \frac{μ mol proline}{g fresh plant} \times \frac{1 mol proline}{1 \times 1 {0}^{6} μ mol proline} \times \frac{0.5 g fresh plant}{1 mL solvent} = \frac{mol proline}{mL solvent} = mM of proline\end{matrix}$$

### Comparison of Amino Acid Concentrations in the Literature

To understand if amino acids other than proline could create a false positive response in our sensors, we converted amino acid concentrations found in the literature to molarity based on the extraction method we use with biological samples. We saw that full amino acid profiles in plants are often reported as a dry plant mass as opposed to fresh mass, which we chose to use for our proline calculations. Apart from this, we followed the same protocol previously described above to convert μmol/g or μg/g values to a unit of molarity.

### Evaluation of Interfering Amino Acids in Sinapaldehyde-Based Sensors

First, we prepared 1 mL samples of the amino acid solutions with increasing amounts of alanine, arginine, aspartic acid, glutamic acid, or leucine (0, 1, 10, 25, and 50 mM) and 50 mM sodium hydroxide in water. We used 6 μL samples to fill the sensors and captured high resolution images for pixel intensity analysis as described above.

Analysis of Interfering Amino Acids with Isatin-Based Sensors

To determine if the paper-based isatin assay was sensitive to other amino acids, we prepared 0.1 M stock solutions of alanine, arginine, aspartic acid, glutamic acid, and leucine in 0.1 M MES buffer (pH=4). We then prepared 1 mL solutions at 0, 1, 10, and 50 mM for each amino acid. We prepared the sensors, ran the assay, and analyzed the results in the same manner described above.

### Analysis of Complex Samples in Sinapaldehyde-Based Sensors

To further explore the effect of interfering amino acids on our sensors, we first made 1 mL aliquots of 0, 2.5, 5, 7.5, and 10 mM solutions of each amino acid (proline, alanine, arginine, aspartic acid, glutamic acid, and leucine) in water with 50 mM NaOH. Additionally, we prepared 1 mL samples that consisted of combinations of proline and another amino acid with a 10 mM total amino acid concentration. These samples consisted of either 2.5 mM proline and 7.5 mM of another amino acid, 5 mM proline and 5 mM of another amino acid, or 7.5 mM proline and 2.5 mM of another amino acid, along with 50 mM NaOH.

Next, we prepared our paper-based sensors with the previously described protocol. We used 6 μL samples to fill the sensors and captured high resolution images for pixel intensity analysis as described above. We also used Microsoft Excel to perform a two-tailed t-test with equal variance where p<0.05. For this statistical analysis, we wanted to only compare the combination samples to the pure proline samples, not to each other, which is why we chose a t-test for this experiment.

### Analysis of Sucrose on Proline Sensors

To deduce if the presence of sugars in the plant extract inhibited the nesocodin formation in our sensors, we prepared stock solutions of 0.1 M proline and 0.2 M sucrose in both water and 3% (w/v) sulfosalicylic acid. We prepared 1 mL samples with 10 mM proline, 0.05 M sodium hydroxide, and concentrations of sucrose ranging from 0 to 150 mM. We applied these samples onto our sensors (see above), allowed the reaction to occur for 15 min, and then took a high-resolution image of each sensor. We reported the results as an average of three sensor replicates and error is the standard deviation.

Proline Extraction from Plants

To test the performance of our sensors using real samples, we measured the proline content of ornamental cabbage and kale plants subjected to intentional, controlled abiotic stresses. In these experiments, we used standardized sampling and extraction methods to evaluate the extent of agreement between the results provided by our paper-based sensors and an established spectrophotometric assay for proline. To test these two measurement methods in parallel, we removed the stalks from sampled plant leaves and then manually ground the remaining material using a spice grinder. We measured the ground samples into separate centrifuge tubes and added extraction matrices of either 100% ethanol (for paper-based sensors) or 3% (w/v) sulfosalicylic acid (for the spectrophotometric acid-ninhydrin assay) at a ratio of 0.5 g of plant material to 1 mL of solution. We vortexed each sample for 1 min and then left them to sit for 3 min. We then used a glass stirring rod to muddle the sample and then pushed the plant sample to the sides of the container and pressed out the solvent. Next, we pipetted the extract mixture into a new centrifuge tube to separate sedimented plant material from the extract. We then analyzed the proline concentration of the ethanol and sulfosalicylic acid extracts using our paper-based sensors and the spectrophotometric ninhydrin assay (see below), respectively.

### Determination of Best-Fit Equation for Calibration Curve

To determine the concentration of proline in samples extracted from plants, we used a modified four-parameter logistic curve fitting equation (Equation 2) to determine the best-fit line for our calibration curve.

In this equation, a is the minimum signal intensity, b is the slope at the inflection point of the curve, c is the inflection point, d is the maximum signal intensity, and x and y are the proline concentration and color intensity in the green channel of the RGB color space, respectively. We normalized the experimental data by subtracting the color intensity of each sample from the average color intensity of the control (Equation 3), and then plotted the experimental data and model data with estimated a, b, c, and d parameters. This was the only case where the color intensities were not subtracted from 255 (explained above) to normalize them. In Equation 3, yₙₒᵣₘ is the normalized color intensity, y₀ is the color intensity with 0 mM proline (control) and yₓ is the color intensity at a given proline concentration (x). We then used Excel Solver to optimize the fitting parameters and produce a best-fit equation with an R2 value of 0.995 (Equation 4).

### Assessing Osmotic Stress in Ornamental Cabbage Plants

We purchased two identical ornamental cabbage plants (Market Basket, Billerica, MA) and allowed them to acclimate indoors for five days. We kept the plants under grow lamps (Halo Grow Light, Lordem) for 12 hours per day. After 5 days, we removed approximately 5 leaves from each plant and measured proline content as described previously. We then left the plants for 24 hours to re-acclimate after removing leaves. Next, we watered one plant with 250 mL of filtered tap water (the control plant) and the other with salt water (the experimental plant). We followed a watering protocol by Pavlovic et al. to introduce the salt to the plant incrementally. [64] In summary, we first watered the plant with 250 mL of 25 mM sodium chloride (NaCl) solution every 2 hours for 8 hours. We then increased the NaCl concentration to 50 mM and added 250 mL of this solution to the plant every 2 hours for 4 hours. We then left the plant to sit for 24 hours. Afterwards, we removed the outer leaves from both plants. We left half of the leaves out to dry for three days, and we placed the other half in airtight bags and stored them in a freezer.

### Assessing Thermal Stress in Ornamental Kale Plants

For this proof-of-concept experiment, we purchased two identical ornamental kale plants (Pond View Gardens, Woburn, MA) and allowed them to acclimate indoors for five days. We kept the kale plants under grow lamps for 12 hours a day. Next, we placed one plant (the experimental plant) in an environmental chamber (HD 205, Associated Environmental Systems) and exposed the plant to a 35° C., 40% humidity environment for 24 hours. We based our choice in temperature off a protocol by Li et al. but chose to increase the suggested time from 12 to 24 hours. [65] After exposure to these conditions, we left the experimental plant to cool for 30 minutes, and then removed 5 outer leaves from both the experimental and control plants. We then placed the experimental plant back in the environmental chamber and ran the same cycle for another 24 hours while the control plant was kept under the grow lamp at ambient temperature. The next day, we removed another 5 outer leaves from each plant. We performed proline extraction immediately after removing leaves from these plants, then performed measurements using our paper-based sensors and the spectrophotometric acid ninhydrin assay.

Proline Analysis with the Ninhydrin Assay

We utilized an acid ninhydrin assay to compare the performance of our sensors to a standard technique. To start, we prepared a proline calibration curve with concentrations ranging from 0.025 to 0.35 mM. We used the protocol described by Bates et al.[40a] In summary, we first prepared acid ninhydrin by dissolving 0.25 g of ninhydrin in 6 mL of glacial acetic acid and 4 mL of 6 M phosphoric acid. Next, we made 1 mL samples of 0.025, 0.05, 0.1, 0.15, 0.2, 0.25, 0.3, and 0.35 mM proline in 3% (w/v) sulfosalicylic acid in glass test tubes and then added 1 mL of glacial acetic acid and 1 mL of the acid ninhydrin solution. We covered the samples and heated them at 100° C. for 1 hour in a water bath. Afterwards, we placed the samples in cold water for 20 min. We removed the samples and added 2 mL of toluene to each one. We vigorously aspirated each sample to thoroughly mix the sample and to transfer the proline into the toluene. We then let the samples sit for 10 min so the toluene could fully separate from the sulfosalicylic acid/acid ninhydrin solution. Afterwards, we pipetted the toluene off the top of the solution and added it into centrifuge tubes. We centrifuged the samples at 10,000 ref for 5 minutes to drive any remaining water to the bottom of the sample.

We analyzed our samples by collecting each of their absorbance profiles on an Ocean Optics spectrophotometer from 300-700 nm. We used a quartz cuvette to measure the samples and measured the peak intensity at 520 nm to develop a calibration curve. Our results are the average of three separate samples and error is the standard deviation.

For the proline analysis of the plant samples, we completed the ninhydrin assay the same day as the proline extraction. For the samples after the first stress event, we added the extract as it was directly to the acetic acid and acid ninhydrin. For the samples after the second stress event, we diluted the extract with 3% (w/v) sulfosalicylic acid by a factor of 4 to prevent the signal from saturating the detector of the spectrophotometer. We executed this assay three times with the same stock of plant extract for each sample and reported the results as the average of the three samples and error as the standard deviation.

Assessing Green Cabbage Samples with Exogenous Proline Additions

To further highlight the selectivity of our sinapaldehyde-based sensors, we first purchased two immature ornamental green cabbage plants (Griggs Farm, Billerica, MA). We imaged the plants and then kept one plant (control) in an ambient environment and placed the other (experimental) in an environmental chamber. We exposed the plant to 35° C., 40% humidity conditions for 24 hours. After the 24 hours, we imaged the plants again and then removed the leaves and performed our proline extraction method with 100% ethanol.

Next, we added equal volumes of the experimental plant extract into four centrifuge tubes, followed by the NaOH solution. We then added increasing amounts of a 50 mM solution of proline in ethanol to three samples so that the extracts would have an additional 2, 6, and 10 mM of proline. We then diluted all of the samples to a final volume of 0.5 mL so the final NaOH concentration was 50 mM and the samples had an additional 0, 2, 6, and 10 mM proline. The control extract was also diluted with 50 mM NaOH and ethanol to replicate the other conditions. We then performed measurements with our sensors in triplicate and measured the concentrations with our calibration curve.

### Statistical Analysis

For all statistical analysis we used Microsoft Excel. For the results in FIG. 11F, 15C, and 16C, we used a two-tailed t-test with equal variance and a p-value<0.05. In FIG. 11F, we compared the colorimetric response of each sample that was a combination of proline and an additional amino acid with the response to proline alone at the matching proline concentration. In FIG. 15C, we compared the results from the control ornamental green cabbage to the experimental one at each condition (before stress, frozen, and dried). In the same manner as in FIG. 15C, in FIG. 16C, the control and experimental kale plants were compared to each other after each stress event.

In FIG. 22C, we chose to compare each measured condition to each other to highlight that the additions of exogenous proline at different concentrations cause distinctly different colorimetric responses. Therefore, we chose to perform a one-way ANOVA test with a p-value<0.05. When our results showed that the p-value was less than 0.05, we then performed a Tukey Test to determine which conditions were significantly different from each other.

### Device Assembly

To ensure ideal alignment of these film and alignment layers, we designed an alignment base made of acrylic (1/8 in. thickness) that we fabricated with a CO₂ laser. Additionally, we laser-cut holes into the plastic films and acrylic layers and assembled devices with plastic alignment pins (1/20 in. diameter) from the bottom of the device to the top layer-by-layer. The base layer of the device was a PET film (0.003 in. thickness) backed with adhesive. We created the next two layers with the same material and backed both sides with adhesive. These layers contained a rectangular vessel rounded on one end to hold the wicking fabric (SHAMWOW® cloth). We used two of these pieces in our devices to accommodate the thickness of the wicking fabric. We then placed the fabric that was cut out with a knife plotter (CRICUT® machine) in the rectangular vessel. To ensure we aligned the fabric correctly, we placed a removable acrylic alignment piece shaped with the same cutout to fit the rounded rectangle of the fabric to easily line up and secure the fabric in place. We designed the alignment pieces to have multiple circular cutouts through them to minimize the surface area of the pieces touching the adhesive sides of the adjacent layers, allowing easy removal. To prepare for the addition of the sinapaldehyde-based sensor, we placed the circular sensor holder and spacer made of PET film and double-sided adhesive onto the exposed wicking material and adhesive of the fabric spacer. We used another alignment piece with a semi-circle cut out on its side to ensure the paper layers were perfectly in contact with the wicking material and made no contact with the adhesive layers. We then placed a 9 mm disc of WHATMAN® 3 filter paper into the circle cut out to promote more uniform sample distribution across the sensors, followed by the sinapaldehyde embedded WHATMAN® 5 sensor. We removed the alignment piece and finished assembling the device by placing the cover and viewing window made of PET film (0.003 in. thickness) backed on one side with adhesive to the device. We laminated the completed device to fully seal the films together and enclose the wicking fabric and sensor.

### Measuring Plant Samples Using Self-Contained Microfluidic Devices

For this experiment, we used the same control and experimental immature green cabbage plants, and the same proline extraction protocol with 100% ethanol as previously described. We then added NaOH to a final concentration of 250 mM to each sample, mixed, and poured the control sample into a shallow dish. We then took a device and submerged the tip of the wicking fabric into the sample and held the device in place until the sensor fully hydrated. We then left the device for 15 minutes to allow the reaction to occur and the color to fully develop. We then captured a high-resolution image of the device using a photo scanner and then repeated the process using extract from the experimental plant.

### Example 2

An example assembly protocol for a 1-part (i.e., 1-sensor) device (FIGS. 1B and 1C).

We assembled the device atop an alignment base (Layer 1) made of acrylic (1/8 in thickness) using a CO₂ laser. To ensure ideal alignment of these film and alignment layers, we laser-cut holes into the plastic films and acrylic layers and assembled devices with plastic alignment pins (1/20 in diameter) from the bottom of the device to the top layer-by-layer. The base layer of the device was a PET film (0.003 in thickness) backed with adhesive (Layer 2). We created a component with a recessed cutout (Layer 3) matching the geometry of an acrylic alignment tool (Layer 4) designed to enable reproducible placement and accommodate the thickness of the fabric wick (Layer 5, ShamWow). With the fabric secure, we removed the alignment piece (Layer 4). To prepare for the addition of the sinapaldehyde-based sensor, we placed the circular sensor holder (Layer 6) made of PET film and double-sided adhesive to the exposed fabric (Layer 5) and adhesive of the fabric holder (Layer 3). We used another alignment piece for the sensor (Layer 7) with a semi-circle cut out on its side to ensure the circular sensor (Layer 8) was perfectly in contact with the fabric wick and did not touch the adhesive layers. We designed both alignment tools (Layers 4 & 7) to have multiple circular cutouts through them to minimize the surface area of the pieces touching the adhesive sides of the adjacent layers, allowing easy removal. We removed the alignment piece (Layer 7) and finished assembling the device by placing the top layer (Layer 9) made of PET film backed on one side with adhesive to the device. We laminated this completed device, and sealed the films together, enclosing the fabric wick and sensor in the device.

An example assembly protocol for a 2- or 3-part device (FIGS. 1A and 1B).

To prepare the water indicator, we first prepared a stock of polyethylene glycol (PEG, 20 kDa) in deionized water. We applied 9 μL of the PEG solution onto a 9 mm disc of WHATMAN® 5 paper. We let the sensors dry in an 80° C. oven for 7 minutes. Next, we prepared a 20% cobalt (11) chloride solution in deionized water. We then fully submerged the discs into the cobalt (11) chloride solution. Then, we placed them in the oven for 7 minutes with the side the PEG was applied to face up. In these designs, there were multiple positions for sensors in Layer 8 of the device, spaced 5 mm apart, and all other device components and alignment tools were designed to accommodate these added features. To make the pH sensor, we applied 7 μL of pH indicator onto 9 mm discs of WHATMAN® 5 paper. We let the sensors dry in a 100° C. oven for 5 minutes.

### REFERENCES

[1] a) U. N. D. o. I. Economic, World population prospects, UN, 1978; b) M. van Dijk, T. Morley, M. L. Rau, Y. Saghai, Nat. Food 2021, 2, 494-501.
[2] a) S. Asseng, F. Ewert, P. Martre, R. P. ROtter, D. B. Lobell, D. Cammarano, B. A. Kimball, M. J. Ottman, G. W. Wall, J. W. White, M. P. Reynolds, P. D. Alderman, P. V. V. Prasad, P. K. Aggarwal, J. Anothai, B. Basso, C. Biernath, A. J. Challinor, G. De Sanctis, J. Doltra, E. Fereres, M. Garcia-Vila, S. Gayler, G. Hoogenboom, L. A. Hunt, R. C. Izaurralde, M. Jabloun, C. D. Jones, K. C. Kersebaum, A. K. Koehler, C. Muller, S. Naresh Kumar, C. Nendel, G. O'Leary, J. E. Olesen, T. Palosuo, E. Priesack, E. Eyshi Rezaei, A. C. Ruane, M. A. Semenov, I. Shcherbak, C. Stackle, P. Stratonovitch, T. Streck, I. Supit, F. Tao, P. J. Thorburn, K. Waha, E. Wang, D. Wallach, J. Wolf, Z. Zhao, Y. Zhu, Nat. Clin. Change 2015, 5, 143-147; b) P. Ciais, M. Reichstein, N. Viovy, A. Granier, J. Ogée, V. Allard, M. Aubinet, N. Buchmann, C. Bernhofer, A. Carrara, F. Chevallier, N. De Noblet, A. D. Friend, P. Friedlingstein, T. Grinwald, B. Heinesch, P. Keronen, A. Knohl, G. Krinner, D. Loustau, G. Manca, G. Matteucci, F. Miglietta, J. M. Ourcival, D. Papale, K. Pilegaard, S. Rambal, G. Seufert, J. F. Soussana, M. J. Sanz, E. D. Schulze, T. Vesala, R. Valentini, Nature 2005, 437, 529-533; c) P. Zhu, J. Burney, J. Chang, Z. Jin, N. D. Mueller, Q. Xin, J. Xu, L. Yu, D. Makowski, P. Ciais, Nat. Clin. Change 2022, 12, 1016-1023.
[3]L. Tian, J. Li, W. Bi, S. Zuo, L. Li, W. Li, L. Sun, Agric. Water Manag. 2019, 218, 250-258.
[4]E. E. Rezaei, H. Webber, S. Asseng, K. Boote, J. L. Durand, F. Ewert, P. Martre, D. S. MacCarthy, Nat. Rev. Earth Environ. 2023, 4, 831-846.
[5]G. S. Malhi, M. Kaur, P. Kaushik, Sustainability 2021, 13, 1318.
[6]P. Högy, H. Wieser, P. Köhler, K. Schwadorf, J. Breuer, J. Franzaring, R. Muntifering, A. Fangmeier, Plant Biol. 2009, 11, 60-69.
[7]K.-H. Erb, C. Lauk, T. Kastner, A. Mayer, M. C. Theurl, H. Haberl, Nat. Commun. 2016, 7, 11382.
[8] a) B. W. Griscom, J. Adams, P. W. Ellis, R. A. Houghton, G. Lomax, D. A. Miteva, W. H. Schlesinger, D. Shoch, J. V. Siikamaki, P. Smith, P. Woodbury, C. Zganjar, A. Blackman, J. Campari, R. T. Conant, C. Delgado, P. Elias, T. Gopalakrishna, M. R. Hamsik, M. Herrero, J. Kiesecker, E. Landis, L. Laestadius, S. M. Leavitt, S. Minnemeyer, S. Polasky, P. Potapov, F. E. Putz, J. Sanderman, M. Silvius, E. Wollenberg, J. Fargione, Proc. Natl. Acad. Sci. U.S.A 2017, 114, 11645-11650; b) S. Roe, C. Streck, M. Obersteiner, S. Frank, B. Griscom, L. Drouet, O. Fricko, M. Gusti, N. Harris, T. Hasegawa, Z. Hausfather, P. Havlík, J. House, G.-J. Nabuurs, A. Popp, M. J. S. Sinchez, J. Sanderman, P. Smith, E. Stehfest, D. Lawrence, Nat. Clin. Change 2019, 9, 817-828; c) D. Lawrence, M. Coe, W. Walker, L. Verchot, K. Vandecar, Front. For. Glob. Change 2022, 5; d) X. Giam, Proc. Natl. Acad. Sci. U.S.A 2017, 114, 5775-5777.
[9]E. C. Oerke, J. Agric. Sci. 2006, 144, 31-43.
[10]S. Savary, A. Ficke, J.-N. Aubertot, C. Hollier, Food Secur. 2012, 4, 519-537.
[11] a) E.-C. Oerke, H.-W. Dehne, Eur. J. Plant Pathol. 1997, 103, 203-215; b) E. C. Oerke, H. W. Dehne, Crop Prot. 2004, 23, 275-285.
[12]Y. Inoue, Soil Sci. Plant Nutr. 2020, 66, 798-810.
[13]M. S. M. Asaari, S. Mertens, S. Dhondt, D. Inzé, N. Wuyts, P. Scheunders, Comput. Electron. Agric. 2019, 162, 749-758.
[14]C. Veys, F. Chatziavgerinos, A. AlSuwaidi, J. Hibbert, M. Hansen, G. Bernotas, M. Smith, H. Yin, S. Rolfe, B. Grieve, Plant Methods 2019, 15, 4.
[15]R. Casari, D. S. Paiva, V. N. B. Silva, T. M. M. Ferreira, M. T. Souza Junior, N. G. Oliveira, A. K. Kobayashi, H. B. C. Molinari, T. T. Santos, R. L. Gomide, P. C. Magalhaes, C. A. F. Sousa, Int. J. Mol. Sci. 2019, 20.
[16] A. V. Zubler, J. Y. Yoon, Biosensors (Basel) 2020, 10.
[17]G. Kitic, A. Tagarakis, N. Cselyuszka, M. Panić, S. Birgermajer, D. Sakulski, J. Matović, Comput. Electron. Agric. 2019, 162.
[18] a) S. Stephen, V. Kumar, J. Indian Soc. Remote Sens. 2023, 51, 2577-2597; b) M. Kalischuk, M. L. Paret, J. H. Freeman, D. Raj, S. Da Silva, S. Eubanks, D. J. Wiggins, M. Lollar, J. J. Marois, H. C. Mellinger, J. Das, Plant Dis. 2019, 103, 1642-1650; c) Y. Guo, X. Jia, D. Paull, J. Zhang, A. Farooq, X. Chen, M. N. Islam, in IGARSS 2019-2019 IEEE International Geoscience and Remote Sensing Symposium, IEEE, 2019, pp. 9376-9379; d) M. F. F. Rahman, S. Fan, Y. Zhang, L. Chen, Agriculture 2021, 11, 22.
[19]N. Coppedè, M. Janni, M. Bettelli, C. L. Maida, F. Gentile, M. Villani, R. Ruotolo, S. Iannotta, N. Marmiroli, M. Marmiroli, A. Zappettini, Sci. Rep. 2017, 7, 16195.
[20]D. C. Tsouros, S. Bibi, P. G. Sarigiannidis, Information 2019, 10, 349.
[21] a) H. Pathak, G. Kumar, S. Mohapatra, B. Gaikwad, J. Rane, ICAR-National Institute of Abiotic Stress Management 2020, 300, 4-15; b) S. C. Hassler, F. Baysal-Gurel, Agronomy 2019, 9, 618.
[22] a) H. M. H. Salama, A. A. Al Watban, A. T. Al-Fughom, Saudi J. Biol. Sci. 2011, 18, 79-86; b) Y. K. Shin, S. R. Bhandari, J. S. Jo, J. W. Song, J. G. Lee, Horticulturae 2021, 7, 238.
[23] a) C. F. Bournonville, J. C. Diaz-Ricci, Phytochem. Anal. 2011, 22, 268-271; b) G. P. Bolwell, P. Wojtaszek, Physiol. Mol. Plant Pathol. 1997, 51, 347-366; c) M. Pinto-Marijuan, S. Munne-Bosch, J. Exp. Bot. 2014, 65, 3845-3857.
[24] a) K. Chaitanya, D. Sundar, A. R. Reddy, Biol. Plant. 2001, 44, 379-384; b) N. M. Barnett, A. W. Naylor, Plant Physiol. 1966, 41, 1222-1230; c) T. Martinelli, A. Whittaker, A. Bochicchio, C. Vazzana, A. Suzuki, C. Masclaux-Daubresse, J. Exp. Bot. 2007, 58, 3037-3046.
[25] a) S. A. Anjum, M. Farooq, X.-y. Xie, X.-j. Liu, M. F. Ijaz, Sci. Hortic. 2012, 140, 66-73; b) D. C. Dien, T. Mochizuki, T. Yamakawa, Plant Prod. Sci. 2019, 22, 530-545.
[26] a) N. Batra, N. Kumari, V. Sharma, Vegetos 2023, 36, 268-274; b) P. Surender Reddy, G. Jogeswar, G. K. Rasineni, M. Maheswari, A. R. Reddy, R. K. Varshney, P. B. Kavi Kishor, Plant Physiol. Biochem. 2015, 94, 104-113.
[27] a) F. Kirbag Zengin, O. Munzuroglu, Acta Biol. Cracov., Ser. Bot. 2005, 47; b) S. S. Sharma, H. Schat, R. Vooijs, Phytochemistry 1998, 49, 1531-1535.
[28]R. M. Rivero, J. M. Ruiz, L. M. Romero, Plant Biol. (Stuttg) 2004, 6, 702-707.
[29]P. P. Saradhi, Alia, S. Arora, K. V. Prasad, Biochem. Biophys. Res. Commun. 1995, 209, 1-5.
[30] a) M. Homayoonzadeh, P. Moeini, K. Talebi, U. Roessner, V. Hosseininaveh, Acta Physiol. Plant. 2020, 42, 161; b) K. Boulahia, P. Carol, S. Planchais, O. Abrous-Belbachir, J. Agric. Food Chem. 2016, 64, 3150-3160.
[31]G. Fabro, I. Kovacs, V. Pavet, L. Szabados, M. E. Alvarez, Mol. Plant Microbe Interact. 2004, 17, 343-350.
[32] a) X. Liang, L. Zhang, S. K. Natarajan, D. F. Becker, Antioxid. Redox Signal 2013, 19, 998-1011; b) S. Hayat, Q. Hayat, M. N. Alyemeni, A. S. Wani, J. Pichtel, A. Ahmad, Plant Signal Behav. 2012, 7, 1456-1466; c) S. Spormann, P. Nadais, F. Sousa, M. Pinto, M. Martins, B. Sousa, F. Fidalgo, C. Soares, Antioxidants (Basel) 2023, 12.
[33]Y. Yoshiba, T. Kiyosue, K. Nakashima, K. Yamaguchi-Shinozaki, K. Shinozaki, Plant Cell Physiol. 1997, 38, 1095-1102.
[34] a) S. Handa, A. K. Handa, P. M. Hasegawa, R. A. Bressan, Plant Physiol. 1986, 80, 938-945; b) S. Dong, Y. Jiang, Y. Dong, L. Wang, W. Wang, Z. Ma, C. Yan, C. Ma, L. Liu, Saudi J. Biol. Sci. 2019, 26, 2006-2017.
[35]F. Anjum, V. Rishi, F. Ahmad, Biochimica et Biophysica Acta (BBA)—Protein Structure and Molecular Enzymology 2000, 1476, 75-84.
[36]R. Govrin, T. Obstbaum, U. Sivan, J. Am. Chem. Soc. 2019, 141, 13311-13314.
[37]J. Matysik, Alia, B. Bhalu, P. Mohanty, Curr. Sci. 2002, 82, 525-532.
[38]P. Hare, W. Cress, Plant Growth Regul. 1997, 21, 79-102.
[39] a) C. Ben Ahmed, B. Ben Rouina, S. Sensoy, M. Boukhriss, F. Ben Abdullah, J. Agric. Food Chem. 2010, 58, 4216-4222; b) H. Shin, S. Oh, D. Kim, J. K. Hong, J. G. Yun, S. W. Lee, K.-H. Son, J. Plant Biotechnol. 2018, 45, 357-363.
[40] a) L. S. Bates, R. P. Waldren, I. D. Teare, Plant Soil 1973, 39, 205-207; b) F. N. Boctor, Anal. Biochem. 1971, 43, 66-70.
[41]M. Oufir, N. Schulz, P. S. Sha Vallikhan, E. Wilhelm, K. Burg, J.-F. Hausman, L. Hoffmann, C. Guignard, J. Chromatogr. A 2009, 1216, 1094-1099.
[42]M. Santhosh, T. Park, Anal. Chim. Acta 2022, 1231, 340399.
[43]D. Long, K. L. Wilkinson, K. Poole, D. K. Taylor, T. Warren, A. M. Astorga, V. Jiranek, J. Agric. Food Chem. 2012, 60, 4259-4264.
[44]R. J. Elliott, D. L. Gardner, Anal. Biochem. 1976, 70, 268-273.
[45]D. J. Grainger, S. Aitken, Clin. Chim. Acta 2004, 343, 113-118.
[46] a) S. Ruhemann, J. Chem. Soc., Trans. 1910, 97, 1438-1449; b) S. Ruhemann, J. Chem. Soc., Trans. 1910, 97, 2025-2031; c) C. B. Bottom, S. S. Hanna, D. J. Siehr, Biochem. Educ. 1978, 6, 4-5.
[47] a) D. D. Van Slyke, R. T. Dillon, D. A. MacFadyen, P. Hamilton, J. Biol. Chem. 1941, 141, 627-669; b) F. P. Chinard, J. Biol. Chem. 1952, 199, 91-95.
[48]Y. Kimura, Anal. Chim. Acta 1967, 37, 240-245.
[49]M. Messer, Anal. Biochem. 1961, 2, 353-359.
[50]W. Troll, J. Lindsley, J. Biol. Chem. 1955, 215, 655-660.
[51]C. Magné, F. Larher, Anal. Biochem. 1992, 200, 115-118.
[52]N. Shabnam, I. Tripathi, P. Sharmila, P. Pardha-Saradhi, Protoplasma 2016, 253, 1577-1582.
[53]Y. S. Choi, M. K. Im, M. R. Lee, C. S. Kim, K. H. Lee, Anal. Chim. Acta 2020, 1105, 169-177.
[54]E. Abrahim, C. Hourton-Cabassa, L. Erdei, L. Szabados, in Plant Stress Tolerance Methods and Protocols (Ed.: R. Sunkar), Human Press, 2010, pp. 317-331.
[55]Y. S. Choi, M. R. Lee, C. S. Kim, K. H. Lee, Rev. Sci. Instrum. 2019, 90, 045002.
[56]R. Roy, N. Moreno, S. A. Brockman, A. Kostanecki, A. Zambre, C. Holl, E. M. Solhaug, A. Minami, E. C. Snell-Rood, M. Hampton, M. A. Bee, Y. Chiari, A. D. Hegeman, C. J. Carter, Proc. Natl. Acad. Sci. U.S.A 2022, 119.
[57]V. Kumar, A. Sharma, R. Kaur, A. K. Thukral, R. Bhardwaj, P. Ahmad, Amino Acids 2017, 49, 821-869.
[58] a) M. Bertamini, L. Zulini, K. Muthuchelian, N. Nedunchezhian, Photosynthetica 2006, 44, 151-154; b) S. Cha-Um, C. Kirdmanee, Pak. J. Bot 2009, 41, 87-98; c) S. Sharma, P. E. Verslues, Plant, Cell Environ. 2010, 33, 1838-1851; d) W. Abu El-Soud, M. M. Hegab, H. AbdElgawad, G. Zinta, H. Asard, Plant Physiol. Biochem. 2013, 71, 173-183; e) M. A. Turan, A. H. A. Elkarim, N. Taban, S. Taban, Afr. J. Agric. Res. 2009, 4, 893-897; f) M. Amirjani, Am. J. Plant Physiol. 2010, 5, 350-360; g) X.-s. WANG, J.-g. HAN, Agric. Sci. China 2009, 8, 431-440.
[59]S. S. Sharma, K.-J. Dietz, J. Exp. Bot. 2006, 57, 711-726.
[60]N. R. Pollock, J. P. Rolland, S. Kumar, P. D. Beattie, S. Jain, F. Noubary, V. L. Wong, R. A. Pohlmann, U. S. Ryan, G. M. Whitesides, Sci. Transl. Med. 2012, 4, 152ra129-152ra129.
[61]C. Sicard, C. Glen, B. Aubie, D. Wallace, S. Jahanshahi-Anbuhi, K. Pennings, G. T. Daigger, R. Pelton, J. D. Brennan, C. D. Filipe, Water Res. 2015, 70, 360-369.
[62] a) B. P. Naidu, L. G. Paleg, D. Aspinall, A. C. Jennings, G. P. Jones, Phytochemistry 1991, 30, 407-409; b) T. Singh, I. Paleg, D. Aspinall, Aust. J. Biol. Sci. 1973, 26, 45-56; c) E. Turhan, A. Eris, Commun. Soil Sci. Plant Anal. 2009, 40, 3308-3322.
[63] a) S. R. Bhandari, C. S. Choi, J. Rhee, J. S. Jo, Y. K. Shin, J. W. Song, J. G. Lee, Chil. J. Agric. Res. 2021, 81, 80+; b) H. Sasaki, K. Ichimura, S. Imada, S. Yamaki, J. Plant Physiol. 2001, 158, 847-852; c) E. Rosa, M. David, M. H. Gomes, J. Sci. Food Agric. 2001, 81, 1145-1149.
[64]I. Pavlović, S. Mlinarić, D. Tarkowská, J. Oklestkova, O. Novák, H. Lepeduš, V. V. Bok, S. R. Brkanac, M. Strnad, B. Salopek-Sondi, Front. Plant Sci. 2019, 10.
[65]Q. Li, G. Zhang, Y. Zhao, L. Gu, Y. Wang, X. Yu, S. Abdullah, Horticulturae 2023, 9, 977.
[66] Djomdi, D.; Glew, R.; Kramer, J.; Hernandez, M.; Ernst, J.; Vanderjagt, D., The amino acid, mineral and fatty acid content of three species of human plant food in Cameroon. Food, Global Science Book 2010, 4, 1-6.
[67] Ingledew, W. M.; Magnus, C. A.; Sosulski, F. W., Influence of Oxygen on Proline Utilization During the Wine Fermentation. American Journal of Enology and Viticulture 1987, 38 (3), 246.
[68] Ough, C. S.; Huang, Z.; An, D.; Stevens, D., Amino Acid Uptake by Four Commercial Yeasts at Two Different Temperatures of Growth and Fermentation: Effects on Urea Excretion and Reabsorption. American Journal of Enology and Viticulture 1991, 42 (1), 26.
[69] Prieto-Blanco, M. C.; Jornet-Martinez, N.; Verdú-Andrés, J.; Molíns-Legua, C.; Campíns-Falcó, P., Quantifying both ammonium and proline in wines and beer by using a PDMS composite for sensoring. Talanta 2019, 198, 371-376.
[70] Son, H.-S.; Hwang, G.-S.; Kim, K. M.; Ahn, H.-J.; Park, W.-M.; Van Den Berg, F.; Hong, Y.-S.; Lee, C.-H., Metabolomic Studies on Geographical Grapes and Their Wines Using 1H NMR Analysis Coupled with Multivariate Statistics. Journal of Agricultural and Food Chemistry 2009, 57 (4), 1481-1490.
[71] Son, H.-S.; Hwang, G.-S.; Ahn, H.-J.; Park, W.-M.; Lee, C.-H.; Hong, Y.-S., Characterization of wines from grape varieties through multivariate statistical analysis of 1H NMR spectroscopic data. Food Research International 2009, 42 (10), 1483-1491.
[72] Truzzi, C.; Annibaldi, A.; Illuminati, S.; Finale, C.; Scarponi, G., Determination of proline in honey: Comparison between official methods, optimization and validation of the analytical methodology. Food Chemistry 2014, 150, 477-481.
[73] Meda, A.; Lamien, C. E.; Romito, M.; Millogo, J.; Nacoulma, O. G., Determination of the total phenolic, flavonoid and proline contents in Burkina Fasan honey, as well as their radical scavenging activity. Food Chemistry 2005, 91 (3), 571-577.
[74] Bogdanov, S.; Martin, P.; Lullmann, C., Harmonised methods of the international honey commission. Swiss Bee Research Centre, FAM, Liebefeld 2002, 5, 1-62.

The teachings of all patents, published applications and references cited herein are incorporated by reference in their entirety.

While this invention has been particularly shown and described with references to examples of embodiments thereof, it will be understood by those skilled in the art that various changes in form and details may be made therein without departing from the scope of the invention encompassed by the appended claims.

What is claimed is:

1. A sensor comprising a conjugated aldehyde.

2. The sensor of claim 1, comprising an α,β-unsaturated aldehyde.

3. The sensor of claim 1, wherein the sensor comprises

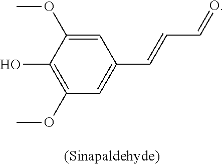

4. The sensor of claim 1, further comprising cellulose.

5. The sensor of claim 1, wherein the sensor has a pore size of less than about 3 μm.

6. The sensor of claim 1, wherein the sensor has a pore size of about 2.5 μm.

7. The sensor of claim 1, wherein the sensor has a diameter of less than about 10 mm.

8. The sensor of claim 1, wherein the sensor has a thickness of from about 0.1 mm to about 1 mm.

9. A device, comprising:
the sensor of claim 1; and
a porous wicking fabric.

10. The device of claim 9, further comprising a substrate comprising cellulose, wherein the substrate is coupled to the sensor and the porous wicking fabric.

11. The device of claim 10, wherein the substrate has a pore size of larger than about 3 μm.

12. The device of claim 10, wherein the substrate has a pore size of about 6 μm.

13. The device of claim 9, further comprising a cover.

14. The device of claim 9, wherein the porous wicking fabric comprising chamois, rayon, cotton, linen, polyester, silk, velvet, or a combination thereof.

15. A kit, comprising:
the sensor of claim 1; and
an extraction solvent.

16. The kit of claim 15, wherein the extraction solvent comprises ethanol or sulfosalicylic acid.

17. The kit of claim 15, further comprising one or more of: a cutting tool, a grinding tool, or a reference tool.

18. A method of determining proline concentration in a plant sample, the method comprising:
contacting the plant sample with the sensor of claim 1 or a device comprising the sensor; and
determining the proline concentration based on color intensity of the sensor or the device.

19. The method of claim 18, wherein determining the proline concentration based on color intensity of the sensor or the device comprises comparing the color intensity of the sensor or the device with a reference tool.

20. A method of analyzing stress in a plant, the method comprising:
contacting a sample of the plant with the sensor of claim 1 or a device comprising the sensor; and
comparing color intensity of the sensor or the device with a reference color intensity to determine stress level of the plant.
